(19) United States
(12) Patent Application Publication
(10) Pub. No.: US 2016/0145599 A1
(43) Pub. Date: May 26, 2016

(54) Mutant Yeasts Having an Increased Production of Lipids and of Citric Acid

(71) Applicants: INSTITUT NATIONAL DE LA RECHERCHE AGRONOMIQUE, Paris (FR); CENTRE NATIONAL DE LA RECHERCHE SCIENTIFIQUE, Paris (FR)

(72) Inventors: Jean-Marc NICAUD, Trappes (FR); Athanasios BEOPOULOS, Paris (FR); Seraphim PAPANIKO-LAOU, Nea Smyrni (GR); Thierry DULERMO, Saint-Germain-en-Laye (FR)

(21) Appl. No.: 14/773,242

(22) PCT Filed: Feb. 28, 2014
(86) PCT No.: PCT/IB2014/059343

§ 371 Date: Sep. 4, 2015

(30) Foreign Application Priority Data
Mar. 4, 2013 (FR) 1351893

(51) Int. Cl. C12N 9/88; C12P 7/48; C12P 7/64

(52) U.S. Cl. CPC C12N 9/88; C12P 7/6463; C12P 7/48; C12Y 402/01079

(57) ABSTRACT

The present invention relates to a mutant yeast strain, in which at least the expression or the activity of the 2-methyl-citrate dehydratase is inhibited, and to the use of said strain for the production of lipids and of citric acid.

The present invention relates to mutant yeasts which exhibit a high production of lipids and of citric acid.

There is currently an overabundance of crude glycerol on the market, which is mainly due to the increasing demand for biodiesel (glycerol being the main by-product of biodiesel production). Likewise, large amounts of aqueous glycerol are generated during the production of bioethanol and/or of alcoholic beverages (for example by fermentation) or during the saponification of fats.

The conversion of glycerol into products with a high added value by means of chemical and/or fermentation technologies is of very great interest.

Some oleaginous microorganisms are capable of converting substrates, such as fats or of glycerol, into lipids, in particular into triglycerides and fatty acids. These oleaginous microorganisms have the capacity to accumulate considerable amounts of lipids, in a proportion of at least 20% of their solids content. In yeasts, a few oleaginous species, termed non-conventional, are found, among which mention may be made of those belonging to the genus Candida, Cryptoccocus, Lipomyces, Rhodosporidium, Rhodotorula, Trichosporon or Yarrowia (for reviews, see Beopoulos et al., 2009a; Papanikolaou et al., 2011a and 2011b).

Yarrowia lipolytica is a hemiascomycete yeast. It is considered to be a model of bioconversion for the production of proteins, enzymes and lipid derivatives (for review, see Nicaud, 2012). It is naturally present in polluted oil environments and in particular in the heavy fractions, thereby attesting to its potential for degrading organic substrates. This yeast has already been successfully tested for its capacity to degrade organic substrates such as naphthalene, dibenzofuran and trinitrotoluene (for review, see: Thevenieau et al., 2009a and 2009b; Beopoulos et al., 2009b and 2009c).

Y. lipolytica is one of the oleaginous yeasts that has been most widely studied owing not only to its capacity to accumulate lipids in a proportion of more than 50% of its solids content according to a defined culture profile, but also to its unique capacity to accumulate linoleic acid at high levels (more than 50% of the fatty acids produced) and also lipids with a high added value, such as stearic acid, palmitic acid and oleic acid, in proportions similar to those found in cocoa butter (Papanikolaou et al., 2001; Papanikolaou et al., 2010).

Y. lipolytica can be efficiently cultured on a large variety of hydrophobic compounds (free fatty acids, triacylglycerols, n-alkanes, etc.), by virtue of the expression of multigene families encoding key enzymes involved in the decomposition of these compounds (for example, acyl-CoA oxidases, lipases). The assimilation of these lipid substrates can lead to a modification of the fatty acid composition both of the residual substrate and of the accumulated fat, sometimes resulting in the synthesis of lipids with advantageous properties (Papanikolaou et al., 2001; Beopoulos et al., 2009a; Papanikolaou et al., 2010; 2011a and 2011b).

Lipid synthesis in Y. lipolytica is carried out either through the de novo biosynthesis of fatty acids via the production of fatty acid precursors such as acetyl-CoA and malonyl-CoA and their integration into the lipid biosynthesis pathway (Kennedy pathway), or through the ex novo accumulation, via the incorporation of the fatty acids pre-existing in the fermentation medium or deriving from the hydrolysis of oils, of fats, of triglycerides and of methyl esters, of the culture medium and their accumulation inside the cell. The main pathways for de novo biosynthesis of lipids in Y. lipolytica and Saccharomyces cerevisiae (S. cerevisiae; yeast referred to as non-oleaginous) are well conserved.

In yeasts, β-oxidation is a fatty acid degradation pathway which is located mainly in the peroxisomes (the biogenesis of which is controlled by the PEX genes). This pathway allows the formation of acetyl-CoA from even-chain fatty acids and of propionyl-CoA from odd-chain fatty acids. β-oxidation comprises four successive reactions during which the carbon-based chain of acyl-CoA is reduced by two carbon atoms. Once the reaction has been carried out, the acyl-CoA reduced by two carbons can return to the β-oxidation spiral (Lynen helix) and undergo a further two-carbon reduction. These decarboxylation cycles can be interrupted depending on the nature of the acyl-CoA, the substrate availability, the presence of coenzyme A and of acetyl-CoA or according to the NAD⁺/NADH ratio. In the first step of β-oxidation, after the release of fatty acids from triacylglycerols (TAGs) by lipases, the active form of acyl-CoA formed is oxidized by a flavin adenine dinucleotide (FAD) molecule so as to form a trans-Δ²-enoyl-CoA molecule by virtue of an acyl-CoA oxidase (AOX). β-oxidation in Y. lipolytica has been widely described (Wang et al., 1999a; Mlickova et al., 2004). There are 6 acyl-CoA oxidases in Y. lipolytica, encoded by the POX1 to 6 genes, which have different substrate specificities (Wang et al., 1999a and 1999b; Luo et al., 2000 and 2002). The trans-Δ²-enoyl-CoA is then hydrolyzed by 2-enoyl-CoA hydratase. The 3-hydroxyacyl-CoA molecule formed is oxidized by NAD so as to form a 3-ketoacyl-CoA molecule. These last two steps are catalyzed by a bifunctional protein encoded by the MFE1 gene (multifunctional protein which has an acyl-CoA hydratase and 3-hydroxyacyl-CoA dehydrogenase activity). The 3-oxoacyl-CoA thioester is then cleaved by a 3-oxoacyl-CoA thiolase encoded by the POT1 gene (Einerhand et al., 1995). A coenzyme A is then added to form an acetyl-CoA and an acyl-CoA reduced by two carbons. Mutant strains of Y. lipolytica in which beta-oxidation of fatty acids is knocked out due to the deletion of the 6 endogenous POX genes have been described by Beopoulos et al. (2008) and in International Application WO 2012/001144.

Various processes have been developed for enabling the fermentation of glycerol by Y. lipolytica and converting it into single cell oil (SCO) and/or into citric acid (citrate) (Papanikolaou et al., 2002; Rywińska et al., 2009; Beopoulos et al., 2009a). The biosyntheses, both of SCO and of citric acid, from glycerol or from other substrates (e.g. hexoses) used as sole substrate or as co-substrate in the first steps of culture, are biochemically equivalent and take place after a nutritional limitation (deficiency) in the culture medium of Y. lipolytica, generally a nitrogen or phosphate limitation (Papanikolaou and Aggelis, 2009; Papanikolaou et al., 2011a). More specifically, for the production of lipids (SCOs), it is necessary to impose a nitrogen limitation by adjusting the C/N ratio with a high concentration of carbon (C) and a low concentration of nitrogen (N), and for the production of citric acid, it is necessary to impose a nitrogen deficiency only. In addition, when Y. lipolytica cell growth is carried out on substrates based on glycerol or on sugar (“monosaccharides”), it is capable either of producing in large amounts only intracellular fats (Tsigie et al., 2011; Fontanille et al., 2012) or of producing only citric acid without accumulating large amounts of cellular lipids (Anastassiadis et al., 2002; Papanikolaou et al., 2002 and 2008; Tai and Stephanopoulos, 2013). It has been reported that, depending on the culture conditions, Y. lipolytica can simultaneously produce citric acid (˜50 g/l) and lipids (˜32% of its solids content) (André et al., 2009), or sequentially produce cellular lipids and citric acid (Makri et al., 2010).

The biosynthesis pathway involved when there is a nitrogen limitation in the culture medium of oleaginous yeasts for lipid production is known (for review, see Beopoulos et al., 2009a). The nitrogen limitation activates AMP deaminase, which leads to a decrease in the concentration of AMP (adenosine monophosphate) in the mitochondria. This decrease in AMP concentration inhibits the isocitrate dehydrogenase enzyme, which catalyzes the conversion of isocitrate to α-ketoglutarate (α-KG). Aconitase catalyzes the isomerization of isocitrate to citrate in the mitochondria. The citrate then leaves the mitochondria and is converted into acetyl-CoA and oxaloacetate by ATP-citrate in the cytosol. The acetyl-CoA accumulated in a large amount in the cytosol allows the synthesis of fatty acid also in a large amount.

Mutant strains of Y. lipolytica (obtained by natural mutation or genetically modified) capable of producing higher amounts of lipids or of citric acid compared with the wild-type strains have been obtained. For example, Rywińska et al. (2009) have obtained acetate-negative mutant strains of Y. lipolytica (ace⁻; incapable of growing on acetate as sole carbon and energy source) capable of bioconverting in batchwise fermentation, glycerol (used as substrate) into citric acid more efficiently than the wild-type strain from which they derive. Tai et al. (2012) have obtained a genetically modified strain of Y. lipolytica overexpressing diacylglycerol acyltransferase (DGA1) and acetyl-CoA carboxylase (ACC1) capable of increasing the production of lipids from glucose, under culture conditions with a high or moderate C/N ratio, compared with the wild-type strain from which it derives. Mutant strains capable of accumulating larger amounts of lipids compared with the wild-type strains have also been described in International Applications WO 2010/004141 and WO 2012/001144.

It therefore appears to be desirable to obtain mutant yeast strains capable of accumulating larger amounts of lipids and/or of citric acid compared with the wild-type strains.

Surprisingly, the inventors have shown that a genetically modified Yarrowia lipolytica yeast strain in which the PHD1 gene (present on chromosome F, YALI0F02497g) encoding 2-methylcitrate dehydratase has been deleted, and which has been cultured on glycerol, exhibits not only a slowed-down consumption of glycerol, but also an increased production of lipids and of citric acid, compared with the wild-type Yarrowia lipolytica yeast strain W29 from which it derives.

In yeasts, 2-methylcitrate dehydratase (nomenclature EC 4.2.1.79) is a mitochondrial protein which catalyzes the conversion of 2-methylcitrate into 2-methyl-cis-aconitate in the 2-methylcitrate cycle of propionate metabolism (Uchiyama et al., 1982).

In particular, in Yarrowia lipolytica, 2-methylcitrate dehydratase is a protein of 520 amino acids, which is encoded by the PHD1 gene (YALI0F02497g). The amino acid sequence of the 2-methylcitrate dehydratase of Y. lipolytica CLIB122 is available under accession number GI:50554999 (or GI:49650778) in the Genbank database, and is represented by the sequence SEQ ID NO: 1. The nucleotide sequence of the cDNA encoding this 2-methylcitrate dehydratase is available under accession number GI:50554998 in the Genbank database.

The inventors have determined that the amino acid sequence of Yarrowia lipolytica 2-methylcitrate dehydratase (SEQ ID NO: 1) has at least 55% identity and at least 70% similarity with the 2-methylcitrate dehydratases of hemiascomycetes, in particular 62% identity and 73% similarity with that of Saccharomyces cerevisiae available under accession number SACE0P06226p in the Génolevures database (Sherman et al., 2009; http://genolevures.org/), 62% identity and 75% similarity with that of Zygosaccharomyces rouxii available under accession number ZYRO0F04466p in the Génolevures database, 61% identity and 75% similarity with that of Saccharomyces kluyveri available under accession number SAKL0B02948p in the Génolevures database, 62% identity and 76% similarity with that of Kluyveromyces lactis var. lactis available under accession number KLLA0E14213p in the Génolevures database, 59% identity and 74% similarity with that of Remothecium gossypii available under accession number ERGO0G08404p in the Génolevures database, 61% identity and 72% similarity with that of Candida glabrata available under accession number CAGL0L09108p in the Génolevures database, 67% identity and 78% similarity with that of Pichia sorbitophila available under accession number PISO0A12716p in the Génolevures database, and 67% identity and 79% similarity with that of Pichia sorbitophila available under accession number PISO0B12783p in the Génolevures database.

The inventors have also determined that the amino acid sequence of Yarrowia lipolytica 2-methylcitrate dehydratase (SEQ ID NO: 1) has at least 85% identity and at least 90% similarity with the 2-methylcitrate dehydratases of strains of Candida of the same Glade as that of Y. lipolytica, in particular 98.7% identity and 99.6% similarity with that of the C. galli CBS9722 strain, 97.9% identity and 99.4% similarity with that of the C. yakushimensis CBS10253 strain, 96.5% identity and 98.1% similarity with that of the C. phangngensis CBS10407 strain, 95.6% identity and 98.5% similarity with that of the C. alimentaria CBS10151 strain, and 87.3% identity and 94% similarity with that of the C. hispaniensis CBS9996 strain.

The inhibition of the expression or of the activity of 2-methylcitrate dehydratase in a yeast makes it possible to obtain a mutant yeast strain capable of producing lipids and citric acid when it is cultured on an appropriate (e.g., glycerol) non-deficient medium.

In addition to the mutation resulting in the inhibition of the expression or of the activity of 2-methylcitrate dehydratase, one or more additional mutations, such as mutations resulting in a fatty acid beta-oxidation deficiency (e.g. inhibition of the endogenous POX1-6, MFE1, POT1 and/or PEX genes), and/or resulting in the accumulation of lipids (e.g., inhibition of the endogenous GUT2 gene and/or overexpression of the endogenous GPD1 gene), and/or resulting in a triglyceride remobilization deficiency (e.g., inhibition of the endogenous TGL3 and/or TGL4 genes) and/or resulting in an increase in lipid production yield (e.g., overexpression of the endogenous ACC1, LRO1 DGA1 and/or DGA2 genes) and/or resulting in an increase in the production of NADPH cofactor for lipid synthesis (e.g., overexpression of the endogenous MAE1 gene) and/or resulting in the production of acetyl-CoA from citrate which is used by FAS (fatty acid synthase) for acyl-CoA synthesis (elongation of the carbon-based chain of fatty acids in the process of being synthesized) (e.g., overexpression of the endogenous ACL1 and ACL2 genes), and/or resulting in the production of acetyl-CoA from acetate which is used by FAS (fatty acid synthase) for acyl-CoA synthesis (elongation of the carbon-based chain of fatty acids in the process of being synthesized) (e.g., overexpression of the endogenous ACS2 gene).

A subject of the present invention is therefore a mutant yeast strain, characterized in that the expression or the activity of the endogenous 2-methylcitrate dehydratase (EC 4.2.1.79) of said strain is inhibited and that, in addition, the expression or the activity of the endogenous acyl-coenzyme A oxidases (EC 6.2.1.3), of the endogenous multifunctional beta-oxidation protein (EC 4.2.1.74), of the endogenous 3-oxoacyl-coenzyme A thiolase (EC 2.3.1.16), of one or more endogenous proteins encoded by a PEX gene involved in yeast peroxisome metabolism (preferably peroxin 10), of one or more endogenous triacylglycerol lipases (EC 3.1.1.3) and/or of the endogenous glycerol 3-phosphate dehydrogenase (EC 1.1.99.5) of said strain is inhibited, and/or one or more of the endogenous genes (preferably all the endogenous genes) encoding a glycerol-3-phosphate dehydrogenase (NAD(+)) (EC 1.1.1.18), and acetyl-CoA carboxylase (EC 6.4.1.2), an acyl-CoA:diacylglycerol acyltransferase (EC 2.3.1.20), an ATP citrate lyase (EC 2.3.3.8), a malic enzyme (EC 1.1.1.40), an acetyl-CoA synthetase (EC 6.2.1.1), a Delta(9)-desaturase (EC 1.14.19.1), a Delta(12)-desaturase (EC 1.14.19.6) and/or an invertase (EC 3.2.1.26) are overexpressed.

Said mutant yeast strain is capable of producing a larger amount of lipids and/or of citric acid than the parent yeast strain from which it derives.

The present invention includes all the yeast strains and in particular the yeast strains belonging to the genus Candida, Cryptoccocus, Hansenula, Kluyveromyces, Lipomyces, Pichia, Rhodosporidium, Rhodotorula, Saccharomyces, Schizzosaccharomyces, Trichosporon or Yarrowia.

Preferably, said yeast strain is an oleaginous yeast strain. Oleaginous yeast strains are well known to those skilled in the art. They have the capacity to accumulate large amounts of lipids, in a proportion of at least 20% of their solids content (see Ratledge, 1994). They generally belong to the genus Candida, Cryptoccocus, Lipomyces, Rhodosporidium (e.g., Rhodosporidium toruloides), Rhodotorula (e.g., Rhodotura glutinis), Trichosporon or Yarrowia.

A strain which is more particularly preferred for the purposes of the present invention is a Yarrowia yeast strain, preferably a Yarrowia lipolytica yeast strain.

Advantageously, said mutant yeast strain is auxotrophic for leucine (Leu⁻) and optionally for orotidine-5′-phosphate decarboxylase (Ura⁻).

The term “2-methylcitrate dehydratase” is intended to mean an enzyme (EC 4.2.1.79) which catalyzes the conversion of 2-methylcitrate into 2-methyl-cis-aconitate and which has at least 55% identity, and in increasing order of preference at least 60%, 65%, 70%, 75%, 80%, 85%, 90%, 91%, 92%, 93%, 94%, 95%, 96%, 97%, 98%, 99% or 100% identity, or 70% similarity, and in increasing order of preference at least 75%, 80%, 85%, 90%, 91%, 92%, 93%, 94%, 95%, 96%, 97%, 98%, 99% or 100% similarity with the amino acid sequence SEQ ID NO: 1, when the sequences are aligned over their entire length.

Unless otherwise specified, the identity and similarity percentages indicated herein are calculated on the basis of an overall alignment of the amino acid sequences, carried out by means of the “needle” algorithm (Needleman and Wunsch, 1970) using the default parameters: “Matrix”: EBLOSUM62, “Gap penalty”: 10.0 and “Extend penalty”: 0.5. The inhibition of the expression or of the activity of 2-methylcitrate dehydratase can be obtained in various ways using methods known in themselves.

Preferably, said 2-methylcitrate dehydratase comprises a prpD region (corresponding to domain PRK09425 in the CDD database: Marchler-Bauer et al., 2011) having the consensus sequence SEQ ID NO: 8 (corresponding to amino acids 37 to 517 of the sequence SEQ ID NO: 1).

In yeasts, the POX1, POX2, POX3, POX4, POX5 and POX6 genes encode respectively 6 isoforms of acyl coenzymeA oxidase (AOX; EC 6.2.1.3) which are at least partially involved in fatty acid β-oxidation. The partial or total inhibition of the expression or of the activity of these isoenzymes results in an increase in lipid accumulation due to the absence of consumption of the lipids synthesized. More particularly, the coding sequence of the POX1 to POX6 genes and the peptide sequence of AOX1 to AOX6 of Y. lipolytica CLIB122 are available in the Génolevures or GenBank databases under the following accession numbers or names: POX1/AOX1=YALI0E32835g/YALI0E32835p, POX2/AOX2=YALI0F10857g/YALI0F10857p; POX3/AOX3=YALI0D24750g/YALI0D24750p; POX4/AOX4=YALI0E27654g/YALI0E27654p; POX5/AOX5=YALI0C23859g/YALI0C23859p; POX6/AOX6=YALI0E06567g/YALI0E06567p. The peptide sequences of the Y. lipolytica acyl-CoA oxidases have 45% identity and 50% similarity with those of the other yeasts. The degree of identity between the acyl-CoA oxidases ranges from 55% to 70% (or 65% to 76% similarity) (International Application WO 2006/064131). A process for inhibiting the expression of the 6 endogenous AOXs in a Y. lipolytica strain has been described in International Applications WO 2006/064131, WO 2010/004141 and WO 2012/001144.

In yeasts, the multifunctional beta-oxidation protein has three domains: two domains which have 3-hydroxyacyl-CoA dehydrogenase activity (EC 4.2.1.74; domains A and B) and one domain which has enoyl-CoA hydratase activity (EC 4.2.1.17; domain C). This enzyme is encoded by the MFE1 (“Multifunctional enzyme type 1”) gene (Haddouche et al., 2011). More particularly, the coding sequence of the MFE1 gene and the peptide sequence of 3-hydroxyacyl-CoA dehydrogenase and enoyl-CoA hydratase of Y. lipolytica CLIB122 are available in the Génolevures or GenBank databases under the following accession number or name: YALI0E15378g/YALI0E15378p. A process for inhibiting the expression of said endogenous multifunctional protein in a Y. lipolytica strain has been described by Haddouche et al. (2011).

In yeasts, 3-oxoacyl-coenzyme A thiolase (EC 2.3.1.16) is encoded by the POT1 (“Peroxisomal Oxoacyl Thiolase 1”) gene (Berninger et al., 1993). More particularly, the coding sequence of the POT1 gene and the peptide sequence of 3-oxoacyl-CoA thiolase of Y. lipolytica CLIB122 are available in the Génolevures or GenBank databases under the following accession number or name: YALI018568g/YALI018568p. A process for inhibiting the expression of endogenous 3-oxoacyl-coenzyme A thiolase in a Y. lipolytica strain has been described by Berninger et al. (1993).

The PEX genes involved in peroxisome metabolism in yeasts, in particular in Y. lipolytica, are described in table 1 below. The coding sequence of the PEX genes is available in the Génolevures or GenBank databases. It has been described in International Application WO 2006/064131 and by Thevenieau et al. (2007) that, when the peroxisome is not correctly assembled or when it is not functional, the fatty acids are not correctly degraded. Advantageously, the expression or the activity of the endogenous peroxin 10 (encoded by the PEX10 gene) of said strain is inhibited.

TABLE 1

| | Accession No. in | Accession No. in | |
|---|---|---|---|
| Gene | S. cerevisiae | Y. lipolytica | Function |
| PEX1 | YKL 197C | YALI0C15356g | AAA-peroxin |
| PEX2 | YJL210W | YALI0F01012g | RING-finger peroxin which functions in |
| | | | peroxisomal matrix protein import |
| PEX3 | YDR329C | YALI0F22539g | Peroxisomal membrane protein (PMP) |
| PEX4 | YGR133W | YALI0E04620g | Peroxisomal ubiquitin conjugating |
| | | | enzyme |
| PEX5 | YDR244W | YALI0F28457g | Peroxisomal membrane signal receptor |
| PEX6 | YNL329C | YALI0C18689g | AAA-peroxin |
| PEX7 | YDR142C | YALI0F18480g | Peroxisomal signal receptor |
| PEX8 | YGR077C | | Intraperoxisomal organizer of the |
| | | | peroxisomal import machinery |
| PEX9 | | YALI0E14729g | Peroxisomal integral membrane protein |
| PEX10 | YDR265W | YALI0C01023g | Peroxisomal membrane E3 ubiquitin |
| | | | ligase |
| PEX11 | YOL147C | YALI0C04092g | Peroxisomal membrane protein |
| PEX12 | YMR026C | YALI0D26642g | C3HC4-type RING-finger peroxisomal |
| | | | membrane peroxin |
| PEX13 | YLR191W | YALI0C05775g | Integral peroxisomal membrane |
| PEX14 | YGL153W | YALI0E9405g | Peroxisomal membrane peroxin |
| PEX15 | YOL044W | | Phosphorylated tail-anchored type II |
| | | | integral peroxisomal membrane protein |
| PEX16 | | YALI0E16599g | Intraperoxisomal peripheral membrane |
| | | | peroxin |
| PEX17 | YNL214W | | Peroxisomal membrane peroxin |
| PEX18 | YHR160C | | Peroxin |
| PEX19 | YDL065C | YALI0B22660g | Chaperone and import receptor |
| PEX20 | | YALI0E06831g | Peroxin |
| PEX21 | YGR239C | | Peroxin |
| PEX22 | YAL055W | | Putative peroxisomal membrane protein |
| PEX23 | PEX30: YLR324w | YALI0D27302g | Integral peroxisomal membrane peroxin |
| | PEX31: YGR004w | | |
| | PEX32: YBR168w | | |
| PEX25 | YPL112C | YALI0D05005g | Peripheral peroxisomal membrane |
| | | | peroxin |
| PEX27 | YOR193W | | Peripheral peroxisomal membrane |
| | | | protein |
| PEX28 | YHR150W | YALI0D11858g | Peroxisomal integral membrane peroxin |
| | | YALI0F19580g | |
| PEX29 | YDR479C | YALI0F19580g | Peroxisomal integral membrane peroxin |
| PEX30 | YLR324W | YALI0D27302g | Peroxisomal integral membrane protein |
| PEX31 | YGR004W | YALI0D27302g | Peroxisomal integral membrane protein |
| PEX32 | YBR168W | YALI0D27302g | Peroxisomal integral membrane protein |

In yeasts, the triacylglycerol lipases (EC 3.1.1.3) are encoded by the TGL genes (Beopoulos et al., 2009 and 2012). Advantageously, the expression or the activity of the triacylglycerol lipase encoded by the TGL3 gene and/or of the triacylglycerol lipase encoded by the TGL4 gene, preferably of the triacylglycerol lipase encoded by the TGL4 gene, is inhibited. The coding sequence of the TGL3 gene and the peptide sequence of the triacylglycerol lipase encoded by the TGL3 gene of Y. lipolytica CLIB122 are available in the Génolevures or GenBank databases under the following accession number or name: YALI0D17534g/YALI0D17534p. The coding sequence of the TGL4 gene and the peptide sequence of the triacylglycerol lipase encoded by the TGL4 gene of Y. lipolytica CLIB122 are available in the Génolevures or GenBank databases under the following accession number or name: YALI0F10010g/YALI0F10010p. A process for inhibiting the expression of an endogenous triacylglycerol lipase in a Y. lipolytica strain has been described in International Application WO 2012/001144 and by Dulermo et al. (2013).

In yeasts, glycerol-3-phosphate dehydrogenase (EC 1.1.99.5) is encoded by the GUT2 gene (Beopoulos et al., 2008). More particularly, the GUT2 gene encodes the Gut2p isoform of glycerol-3-phosphate dehydrogenase, which catalyzes the reaction of oxidation of glycerol-3-phosphate to DHAP (“glycerol dehydratase-reactivation factor”) (Beopoulos et al., 2008). The coding sequence of the GUT2 gene and the peptide sequence of the glycerol-3-phosphate dehydrogenase of Y. lipolytica CLIB122 are available in the Génolevures or GenBank databases under the following accession number or name: YALI0B13970g/YALI0B13970p. A process for inhibiting the expression of said endogenous glycerol-3-phosphate dehydrogenase in a Y. lipolytica strain has been described in International Applications WO 2010/004141 and WO 2012/001144 and by Beopoulos et al. (2008).

In yeasts, glycerol-3-phosphate dehydrogenase (NAD(+)) (EC 1.1.1.18) is encoded by the GPD1 gene (Dulermo et al., 2011). More particularly, the coding sequence of the GPD1 gene and the peptide sequence of the glycerol-3-phosphate dehydrogenase (NAD(+)) of Y. lipolytica CLIB122 are available in the Génolevures or GenBank databases under the following accession number or name: YALI0B02948g/YALI0B02948p. A process for overexpressing endogenous glycerol-3-phosphate dehydrogenase (NAD(+)) in a Y. lipolytica strain has been described in International Application WO 2012/001144.

In yeasts, acetyl-CoA carboxylase (EC 6.4.1.2) is encoded by the ACC1 gene (Tai et al., 2012, Beopoulos et al., 2012). More particularly, the coding sequence of the ACC1 gene and the peptide sequence of the acetyl-CoA carboxylase of Y. lipolytica CLIB122 are available in the Génolevures or GenBank databases under the following accession number or name: YALI0C11407g/YALI0C11407p. A process for overexpressing endogenous acetyl-CoA carboxylase in a Y. lipolytica strain has been described by Thai et al. (2012).

In yeasts, acyl-CoA:diacylglycerol acyltransferases (DGAT; EC 2.3.1.20) are encoded by two genes: DGA1 and DGA2 (Beopoulos et al., 2009 and 2012; Tai et al., 2012; International Application WO 2012/001144). More particularly, the coding sequence of the DGA1 gene and the peptide sequence of the acyl-CoA:diacylglycerol acyltransferase 1 of Y. lipolytica CLIB122 are available in the Génolevures or GenBank databases under the following accession number or name: YALI0E32769g/YALI0E32769p. The coding sequence of the DGA2 gene and the peptide sequence of the acyl-CoA:diacylglycerol acyltransferase 2 of Y. lipolytica CLIB122 are available in the Génolevures or GenBank databases under the following accession number or name: YALI0D07986g/YALI0D07986p. In Rhodoturula glutanis, an acyl-CoA:diacylglycerol acyltransferase has been described by Rani et al. (2013). A process for overexpressing one or the two endogenous DGATs (DGAT1 and/or DGAT2) in a Y. lipolytica strain has been described by Beopoulos et al. (2012) and by Tai et al. (2012). Advantageously, the DGA2 gene is overexpressed in the strain according to the invention.

In yeasts, ATP citrate lyase (E.C. 2.3.3.8) consists of two subunits (A and B) encoded by two genes (ACL1 and ACL2, respectively) (Beopoulos et al., 2009). The ATP citrate lyase of certain oleaginous yeasts has been characterized by Boulton et al. (1981). More particularly, the coding sequence of the ACL1 and ACL2 genes and the peptide sequence of subunits A and B of the ATP citrate lyase of Y. lipolytica CLIB122 are available in the Génolevures or GenBank databases under the following accession numbers or names: ACL1/subunit A: YALI0E34793g/YALI0E347939p, and ACL2/subunit B: YALI0D24431g/YALI0D24431p. A process for overexpressing endogenous ATP citrate lyase in a Y. lipolytica strain has been described by Zhou et al. (2012).

In yeasts, malic enzyme (EC 1.1.1.40) is encoded by the MAE1 gene (Beopoulos et al., 2009a). More particularly, the coding sequence of the MAE1 gene and the peptide sequence of the malic enzyme of Y. lipolytica CLIB122 are available in the Génolevures or GenBank databases under the following accession number or name: YALI0E18634g/YALI0E18634p. A process for overexpressing endogenous malic enzyme in a Y. lipolytica strain has been described by Zhang et al. (2013).

In yeasts, phospholipid:diacylglycerol acyltransferase (PDAT; EC 2.3.1.158), encoded by the LRO1 gene, is an enzyme capable of catalyzing the formation of triacylglycerol from 1,2-sn-diacylglycerol (Beopoulos et al., 2009 and 2012). More particularly, the coding sequence of the LRO1 gene and the peptide sequence of the phospholipid:diacylglycerol acyltransferase of Y. lipolytica CLIB122 are available in the Génolevures or GenBank databases under the following accession number or name: YALI0E16797g/YALI0E16797p. A process for overexpressing endogenous phospholipid:diacylglycerol acyltransferase in a Y. lipolytica strain has been described by Beopoulos et al. (2012).

In yeasts, acetate-CoA ligase, acetyl-CoA synthetase (EC 6.2.1.1), acyl-CoA synthetases and coumarate-CoA ligases (EC 6.2.1.12) are proteins belonging to the Génolevure family GL3C0072 composed of 39 genes which are encoded by the genes of which the peptide sequences are available in the Génolevures database under the accession numbers SACE0A00462p, SACE0B07502p, SACE0L04796p, CAGL0B02717p, CAGL0K06853p, CAGL0L00649p, ZYRO0C00682p, ZYRO0E01936p, ZYRO0F14410p, SAKL0A06996p, SAKL0D14608p, SAKL0H14542p, KLTH0G11198p, KLTH0H06490p, KLLA0A03333p, KLLA0D17336p, ERGO0A08558p, ERGO0D18634p, ERGO0G04994p, DEHA2D12606p, DEHA2E05676p, PISO0K02452p, PISO0K15036p, PISO0L02453p, PISO0L15037p, YALI0A14234p, YALI0A15103p, YALI0B05456p, YALI0B07755p, YALI0C05885p, YALI0C09284p, YALI0D17314p, YALI0E05951p, YALI0E11979p, YALI0E12419p, YALI0E12859p, YALI0E20405p, YALI0F05962p and YALI0F06556p. Acetyl-CoA synthetase (EC 6.2.1.1) belongs to the Génolevures family GL3C0072. It is encoded by the ACS2 gene. More particularly, the coding sequence of the ACS2 gene and the peptide sequence of the acetyl-CoA synthetase of Y. lipolytica CLIB122 are available in the Génolevures or GenBank databases under the following accession number or name: YALI0F05962g/YALI0F05962p. The overexpression of ACS2 makes it possible to increase the acetyl-CoA pool. A process for overexpressing endogenous acetyl-CoA synthetase in a Y. lipolytica strain has been described by Zhou et al., (2012).

In yeasts, Delta(9)-desaturase (EC 1.14.19.1) is encoded by the OLE1 gene (Thevenieau and Nicaud, 2013). More particularly, the coding sequence of the OLE1 gene and the peptide sequence of Delta(9)-desaturase of Y. lipolytica CLIB122 are available in the Génolevures or GenBank databases under the following accession number or name: YALI0C05951g/YALI0C05951p. The overexpression of OLE1 makes it possible to enrich the produced oil with C18:1₍ₙ₋₉₎.

In yeasts, Delta(12)-desaturase (EC 1.14.19.6) is encoded by the FAD2 gene (Beopoulos et al., 2014). More particularly, the coding sequence of the FAD2 gene and the peptide sequence of the Delta(12)-desaturase of Y. lipolytica CLIB122 are available in the Génolevures or GenBank databases under the following accession number or name: YALI0B10153g/YALI0B10153p. The overexpression of FAD2 makes it possible to enrich the produced oil with C18:2₍ₙ₋₆₎. A process for overexpressing the Delta(12)-desaturase of Mortierella alpina in a Y. lipolytica strain has been described by Chuang et al. (2009).

In yeasts, invertase (EC 3.2.1.26) is encoded by the SUC2 gene (Lazar et al., 2013). More particularly, the coding sequence of the SUC2 gene and the peptide sequence of the invertase of S. cerevisiae are available in the Uniprot or GenBank databases under the following accession number or name: P00724/YIL162W. The overexpression of SUC2 allows the use of pure sucrose and of molasses (Lazar et al., 2013). A process for overexpressing endogenous acetyl-CoA synthetase in an S. cerevisiae strain has been described by Chen et al. (2010).

A strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase of said strain (in particular the 2-methylcitrate dehydratase encoded by the PHD1 gene in the case of Yarrowia) is inhibited, and the β-oxidation of the fatty acids of said strain is also inhibited. The inhibition of the β-oxidation of the fatty acids of said strain can be carried out by inhibiting the expression or the activity of all the endogenous isoforms of acyl-coenzymeA oxidase of said strain (in particular the 6 isoforms of acyl-coenzymeA oxidase that are encoded by the POX1 to POX6 genes in the case of Yarrowia) and/or by inhibiting the expression or the activity of the endogenous multifunctional beta-oxidation protein of said strain (in particular the multifunctional beta-oxidation protein encoded by the MFE1 gene in the case of Yarrowia) and/or by inhibiting the expression or the activity of the endogenous 3-oxoacyl-coenzyme A thiolase of said strain (in particular the 3-oxoacyl-coenzyme A thiolase encoded by the POT1 gene in the case of Yarrowia) and/or by inhibiting the expression or the activity of one or more proteins encoded by the PEX genes involved in yeast peroxisome metabolism. Preferably, the inhibition of the β-oxidation of the fatty acids of said strain is obtained by inhibiting the expression or the activity of all the endogenous isoforms of acyl-coenzymeA oxidase of said strain (in particular the 6 isoforms of acyl-coenzymeA oxidase that are encoded by the POX1 to POX6 genes in the case of Yarrowia) and/or by inhibiting the expression or the activity of the endogenous multifunctional beta-oxidation protein of said strain (in particular the multifunctional beta-oxidation protein encoded by the MFE1 gene in the case of Yarrowia).

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited. An example of such a strain is the Y. lipolytica strain JMY3433 described hereinafter.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia) and a glycerol-3-phosphate dehydrogenase (NAD(+)) (in particular the GPD1 gene in the case of Yarrowia) are overexpressed. An example of such a strain is the Y. lipolytica strain JMY3776 described hereinafter.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia) and an ATP citrate lyase (in particular the ACL1 and ACL2 genes in the case of Yarrowia) are overexpressed. An example of such a strain is the Y. lipolytica strain JMY4079 described hereinafter.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), a malic enzyme (in particular the MAE1 gene in the case of Yarrowia) and an acetyl-CoA carboxylase (in particular the ACC1 gene in the case of Yarrowia) are overexpressed. An example of such a strain is the Y. lipolytica JMY4209 strain described hereinafter.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia), of the endogenous multifunctional beta-oxidation protein (in particular the multifunctional beta-oxidation protein encoded by the MFE1 gene in the case of Yarrowia) and of one or more endogenous peroxins, such as peroxin 10 (in particular the peroxin 10 encoded by the PEX10 gene in the case of Yarrowia), of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia) and an ATP citrate lyase (in particular the ACL1 and ACL2 genes in the case of Yarrowia) are overexpressed.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia) and an acetyl-CoA synthetase (in particular the ACS2 gene in the case of Yarrowia) are overexpressed.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), an acetyl-CoA synthetase (in particular the ACS2 gene in the case of Yarrowia) and a Delta(9)-desaturase and/or a Delta(12)-desaturase (in particular the OLE1 and/or FAD2 genes respectively in the case of Yarrowia) are overexpressed.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), an acetyl-CoA synthetase (in particular the ACS2 gene in the case of Yarrowia), a Delta(9)-desaturase and/or a Delta(12)-desaturase (in particular the OLE1 and/or FAD2 genes respectively in the case of Yarrowia) and an invertase (in particular the SUC2 gene in the case of Yarrowia) are overexpressed.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase (in particular the 2-methylcitrate dehydratase encoded by the PHD1 gene in the case of Yarrowia), of one or more endogenous triacylglycerol lipases (in particular the triacylglycerol lipase encoded by the TGL4 gene in the case of Yarrowia) and of the endogenous multifunctional beta-oxidation protein (in particular the multifunctional beta-oxidation protein encoded by the MFE1 gene in the case of Yarrowia) of said strain is inhibited, and the endogenous genes encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), a malic enzyme (in particular the MAE1 gene in the case of Yarrowia), an acetyl-CoA synthetase (in particular the ACS2 gene in the case of Yarrowia) and an acetyl-CoA carboxylase (in particular the ACC1 gene in the case of Yarrowia) are overexpressed.

Another strain which is advantageous for the purposes of the present invention is a mutant yeast strain, preferably a mutant Yarrowia strain, more preferably a mutant Yarrowia lipolytica strain, in which the expression or the activity of the endogenous 2-methylcitrate dehydratase of said strain is inhibited, the expression of the endogenous TGL4 gene of said strain is inhibited and the endogenous GPD1 and ACC1 genes of said strain are overexpressed.

The inhibition of the expression or of the activity of an enzyme defined in the present invention may be total or partial. It may be obtained in various ways using methods known in themselves to those skilled in the art.

Advantageously, this inhibition may be obtained by mutagenesis of the gene encoding said enzyme.

The mutagenesis of the gene encoding said enzyme can occur at the level of the coding sequence or of the sequences for regulating the expression of this gene, in particular the level of the promoter, resulting in an inhibition of the transcription or of the translation of said enzyme.

Advantageously, with regard to the inhibition by mutagenesis of the gene encoding the 2-methylcitrate dehydratase, the mutation at the level of the coding sequence is carried out in the sequence encoding the prpD region of the 2-methylcitrate dehydratase.

The mutagenesis of the gene encoding said enzyme can be carried out by genetic engineering. The deletion of all or part of said gene and/or the insertion of an exogenous sequence may, for example, be carried out. Methods for deleting or inserting a given genetic sequence in yeast, in particular in Y. lipolytica, are well known to those skilled in the art (for review, see Madzak et al., 2004). By way of example, use may be made of the method called POP IN/POP OUT which has been used in yeasts, particularly in Y. lipolytica, for deletion of the LEU2, URA3 and XPR2 genes (Barth and Gaillardin, 1996). Use may also be made of the SEP method (Maftahi et al., 1996) which has been adapted in Y. lipolytica for detection of the PDX genes (Wang et al., 1999a). Advantageously, use may also be made of the SEP/Cre method developed by Fickers et al. (2003) and described in International Application WO 2006/064131. In addition, methods which make it possible to inhibit the expression or the activity of a yeast enzyme (or protein) are described in International Application WO 2012/001144. A very advantageous method according to the present invention consists in replacing the coding sequence of the gene encoding said enzyme with an expression cassette containing the sequence of a gene encoding a selectable marker (e.g., the URA3 gene [YALI0E26719g] encoding orotidine-5′-phosphate decarboxylase). It is also possible to introduce one or more point mutations into the gene encoding said enzyme, resulting in a shift of the reading frame and/or the introduction of a stop colon into the sequence and/or inhibition of the transcription or the translation of the gene encoding said enzyme.

The mutagenesis of the gene encoding said enzyme may also be carried out using physical agents (for example radiation) or chemical agents. This mutagenesis also makes it possible to introduce one or more point mutations into the gene encoding said enzyme.

The mutated gene encoding said enzyme can be identified, for example, by PCR using primers specific for said gene.

It is possible to use any selection method known to those skilled in the art which is compatible with the marker gene (or genes) used. The selectable markers which allow complementation of an auxotrophy, also commonly called auxotrophic markers, are well known to those skilled in the art. The URA3 selectable marker is well known to those skilled in the art. More specifically, a yeast strain in which the URA3 gene (sequence available in the Génolevures database under the name YALI0E26741g or UniProt database under accession number Q12724), encoding orotidine-5′-phosphate decarboxylase, is inactivated (for example by deletion), will not be capable of growing on a medium not supplemented with uracil. The integration of the URA3 selectable marker into this yeast strain will then make it possible to restore the growth of this strain on a uracil-free medium. The LEU2 selectable marker described in particular in patent U.S. Pat. No. 4,937,189 is also well known to those skilled in the art. More specifically, a yeast strain in which the LEU2 gene (YALI0C00407g), encoding β-isopropylmalate dehydrogenase, is inactivated (for example by deletion) will not be capable of growing on a medium not supplemented with leucine. As previously, the integration of the LEU2 selectable marker into this yeast strain will then make it possible to restore the growth of this strain on a medium not supplemented with leucine. The ADE2 selectable marker is also well known to those skilled in the art in the field of yeast transformation. A yeast strain in which the ADE2 gene (YALI0B23188g), encoding phosphoribosylaminoimidazole carboxylase, is inactivated (for example by deletion) will not be capable of growing on a medium not supplemented with adenine. Here again, the integration of the ADE2 selectable marker into this yeast strain will then make it possible to restore the growth of this strain on a medium not supplemented with adenine. Auxotrophic Leu⁻ Ura⁻Y. lipolytica strains have been described by Barth and Gaillardin, 1996. Auxotrophic Leu⁻ Ura⁻ Ade⁻Y. lipolytica strains have been described in particular in Application WO 2009/098263.

A subject of the present invention is also a process for obtaining a mutant yeast strain according to the present invention from a parent yeast strain, comprising a step of mutagenesis of the gene encoding the 2-methylcitrate dehydratase as defined above in said parent yeast strain, and also one or more steps of mutagenesis in said parent yeast strain resulting in the inhibition of one or more of the endogenous genes encoding the acyl-coenzyme A oxidases (EC 6.2.1.3), the multifunctional beta-oxidation protein (EC 4.2.1.74), the 3-oxoacyl-coenzyme A thiolase (EC 2.3.1.16), the proteins encoded by the PEX genes involved in yeast peroxisome metabolism (preferably peroxin 10), the triacylglycerol lipases (EC 3.1.1.3) and/or the glycerol-3-phosphate dehydrogenase (EC 1.1.99.5) (in particular the POX1 to POX6, MFE1, POT1, PEX, PEX10, TGL3, TGL4 and GUT2 genes in the case of Yarrowia), and/or a step of mutagenesis in said parent yeast strain resulting in the overexpression of one or more of the endogenous genes encoding a glycerol-3-phosphate dehydrogenase (NAD(+)) (EC 1.1.1.18), an acetyl-CoA carboxylase (EC 6.4.1.2), an acyl-CoA:diacylglycerol acyltransferase (EC 2.3.1.20), an ATP citrate lyase (EC 2.3.3.8), a malic enzyme (EC 1.1.1.40), an acetyl-CoA synthetase (EC 6.2.1.1), a Delta(9)-desaturase (EC 1.14.19.1), a Delta(12)-desaturase (EC 1.14.19.6) and/or an invertase (EC 3.2.1.26) (in particular the GPD1, ACC1, DGA1, DGA2, ACL1, ACL2, MAE1, ACS2, OLE1, FAD2 and/or SUC2 genes, in the case of Yarrowia).

According to advantageous embodiments of the process for obtaining a mutant yeast strain according to the present invention, said process comprises:
steps of mutagenesis resulting in the inhibition of the genes encoding the endogenous 2-methylcitrate dehydratase of said strain (in particular the 2-methylcitrate dehydratase encoded by the PHD1 gene in the case of Yarrowia) and in the inhibition of the fatty acid β-oxidation of said strain. The inhibition of the fatty acid β-oxidation of said strain can be carried out as described above; or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL3 or TGL4 gene, preferably the TGL4 gene, in the case of Yarrowia) and the multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an endogenous acyl-CoA:diacylglycerol acyltransferase (in particular the DGA2 gene in the case of Yarrowia) and a glycerol-3-phosphate dehydrogenase (NAD(+)) (in particular the GPD1 gene in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL3 or TGL4 gene, preferably the TGL4 gene, in the case of Yarrowia) and the multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an endogenous acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia) and an ATP citrate lyase (in particular the ACL1 and ACL2 genes in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL3 or TGL4 gene, preferably the TGL4 gene, in the case of Yarrowia) and the multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), a malic enzyme (in particular the MAE1 gene in the case of Yarrowia) and an acetyl-CoA carboxylase (in particular the ACC1 gene in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL4 gene in the case of Yarrowia), the multifunctional beta-oxidation protein (in particular the multifunctional beta-oxidation protein encoded by the MFE1 gene in the case of Yarrowia) and one or more peroxins such as peroxin 10 (in particular the peroxin 10 encoded by the PEX10 gene in the case of Yarrowia), and steps of mutagenesis resulting in the overexpression of one or more of the endogenous genes of said strain encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia) and an ATP citrate lyase (in particular the ACL1 and ACL2 genes in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL4 gene in the case of Yarrowia), the multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia) and an acetyl-CoA synthetase (in particular the ACS2 gene in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL4 gene in the case of Yarrowia) and the multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), an acetyl-CoA synthetase (in particular the ACS2 gene in the case of Yarrowia), a Delta(9)-desaturase and/or a Delta(12)-desaturase (in particular the OLE1 and/or FAD2 genes respectively in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more triacylglycerol lipases (in particular the triacylglycerol lipase encoded by the TGL4 gene in the case of Yarrowia) and the multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an acyl-CoA:diacylglycerol acyltransferase (in particular the DGA2 gene in the case of Yarrowia), a glycerol-3-phosphate dehydrogenase (NAD(+)) (in particular the GPD1 gene in the case of Yarrowia), an ATP citrate lyase (in particular the ACL1 and ACL2 genes in the case of Yarrowia), an acetyl-CoA synthetase (in particular the ACS2 gene in the case of Yarrowia), a Delta(9)-desaturase and/or a Delta(12)-desaturase (in particular the OLE1 and/or FAD2 genes respectively in the case of Yarrowia), and an invertase (in particular the SUC2 gene in the case of Yarrowia); or
steps of mutagenesis resulting in the inhibition of the endogenous genes of said strain encoding 2-methylcitrate dehydratase (in particular the 2-methylcitrate dehydratase encoded by the PHD1 gene in the case of Yarrowia), one or more endogenous triacylglycerol lipases (in particular the triacylglycerol lipase encoded by the TGL4 gene in the case of Yarrowia) and the endogenous multifunctional beta-oxidation protein (in particular the multifunctional beta-oxidation protein encoded by the MFE1 gene in the case of Yarrowia), and steps of mutagenesis resulting in the overexpression of one or more of the endogenous genes of said strain encoding an acyl-CoA:diacylglycerol acyltransferase (in particular the DGA2 gene in the case of Yarrowia), a glycerol-3-phosphate dehydrogenase (NAD(+)) (NAD(+)) (in particular the GPD1 gene in the case of Yarrowia), an ATP citrate lyase (in particular the ACL1 and ACL2 genes in the case of Yarrowia), a malic enzyme (in particular the MAE1 gene in the case of Yarrowia), an acetyl-CoA synthetase (in particular the ACS2 gene in the case of Yarrowia) and an acetyl-CoA carboxylase (in particular the ACC1 gene in the case of Yarrowia); or
steps of mutagenesis resulting in the inhibition of the genes encoding the endogenous 2-methylcitrate dehydratase of said strain (in particular the 2-methylcitrate dehydratase encoded by the PHD1 gene in the case of Yarrowia) and of the endogenous TGL4 gene and a step of mutagenesis resulting in the overexpression of the endogenous GPD1 and ACC1 genes.

The inhibition and/or the overexpression of the endogenous genes can be carried out by genetic engineering.

Said parent yeast strain may be a wild-type yeast strain (e.g., the Y. lipolytica strain W29) or a mutant yeast strain (e.g., the Y. lipolytica strain Pold).

According to one advantageous embodiment of this process, the mutagenesis step comprises the deletion of the coding sequence of the gene encoding a given enzyme (e.g., 2-methylcitrate dehydratase) and optionally replacement of this coding sequence with an exogenous sequence, such as, for example, the sequence of a gene encoding a selectable marker (e.g., the URA3 gene).

A subject of the present invention is also a process for increasing the lipid and/or citric acid production of a yeast strain, characterized in that the expression or the activity of 2-methylcitrate dehydratase is inhibited in said yeast strain.

The inhibition of the expression or of the activity of 2-methylcitrate dehydratase can be carried out as described above.

According to one advantageous embodiment, the process for increasing the lipid production also comprises the inhibition, in said yeast strain, of the expression of one or more of the endogenous genes encoding the acyl-coenzyme A oxidases (EC 6.2.1.3), the multifunctional beta-oxidation protein (EC 4.2.1.74), the 3-oxoacyl-coenzyme A thiolase (EC 2.3.1.16), the proteins encoded by the PEX genes involved in yeast peroxisome metabolism, in particular peroxin 10, the triacylglycerol lipases (EC 3.1.1.3) and/or the glycerol-3-phosphate dehydrogenase (EC 1.1.99.5) (in particular the POX1 to POX6, MFE1, POT1, PEX, TGL3, TGL4 and GUT2 genes in the case of Yarrowia) and/or the overexpression, in said yeast strain, of one or more of the endogenous genes encoding a glycerol-3-phosphate dehydrogenase (NAD(+)) (EC 1.1.1.18), an acetyl-CoA carboxylase (EC 6.4.1.2), an acyl-CoA:diacylglycerol acyltransferase (EC 2.3.1.20), an ATP citrate lyase (EC 2.3.3.8), a malic enzyme (EC 1.1.1.40), an acetyl-CoA synthetase (EC 6.2.1.1), a Delta(9)-desaturase (EC 1.14.19.1), a Delta(12)-desaturase (EC 1.14.19.6) and/or an invertase (EC 3.2.1.26) (in particular the GPD1, ACC1, DGA1, DGA2, ACL1, ACL2, MAE1, ACS2, OLE1, FAD2 and/or SUC2 genes in the case of Yarrowia).

A subject of the present invention is also the use of a mutant yeast strain in which the expression or the activity of the endogenous 2-methylcitrate dehydratase (EC 4.2.1.79) of said strain is inhibited, for the production of lipids and/or of citric acid.

Advantageously, a mutant yeast strain according to the present invention as defined above is used for the production of lipids and/or of citric acid.

The production of lipids can be favored over the production of citric acid when the mutant yeast strain according to the present invention is cultured while controlling the value of the ratio of the rate of carbon consumption to the rate of nitrogen consumption, as described in International Application WO 2010/076432. The production of lipids can also be favored over the production of citric acid by using a mutant strain according to the present invention overexpressing the genes encoding ATP citrate lyase, ACC (acetyl-CoA carboxylase), DGA1 (diacylglycerol acyltransferase 1) and/or DGA2 (diacylglycerol acyltransferase 2). Methods for promoting the accumulation of lipids are also described by Beopoulos et al. (2009).

A subject of the present invention is also a process for producing lipids and/or citric acid, comprising a step of culturing a mutant yeast strain in which the expression or the activity of the endogenous 2-methylcitrate dehydratase (EC 4.2.1.79) of said strain is inhibited, on an appropriate medium.

Advantageously, the process for producing lipids and/or citric acid, comprises a step of culturing a mutant yeast strain according to the present invention as defined above on an appropriate medium.

The methods for extracting the lipids and/or the citric acid that are produced by yeasts in culture are well known to those skilled in the art (Papanikolaou et al., 2001; 2002 and 2008; André et al., 2009). By way of example, the total lipids (extracted in a Folch mixture) can be extracted according to the method described by Papanikolaou et al., 2001, and fractionated according to the methods described by Guo et al., 2000 and Fakas et al., 2006, while the organic acids produced and the residual glycerol can typically be purified by high performance liquid chromatography (HPLC).

According to one preferred embodiment of this process, the medium contains glucose and/or glycerol as carbon source; preferably the medium contains only glycerol as carbon source.

The glycerol may be crude or pure.

Advantageously, said medium is not deficient in nitrogen.

The production of lipids can be favored over the production of citric acid as indicated above.

The present invention will be understood more clearly by means of the additional description which follows, which refers to a nonlimiting example illustrating the increase in the production of lipids and of citric acid by a mutant Y. lipolytica yeast strain in which the expression of 2-methylcitrate dehydratase is inhibited (strain JMY1203), compared with the wild-type Y. lipolytica strain W29 from which it derives, and also of the appended figures:

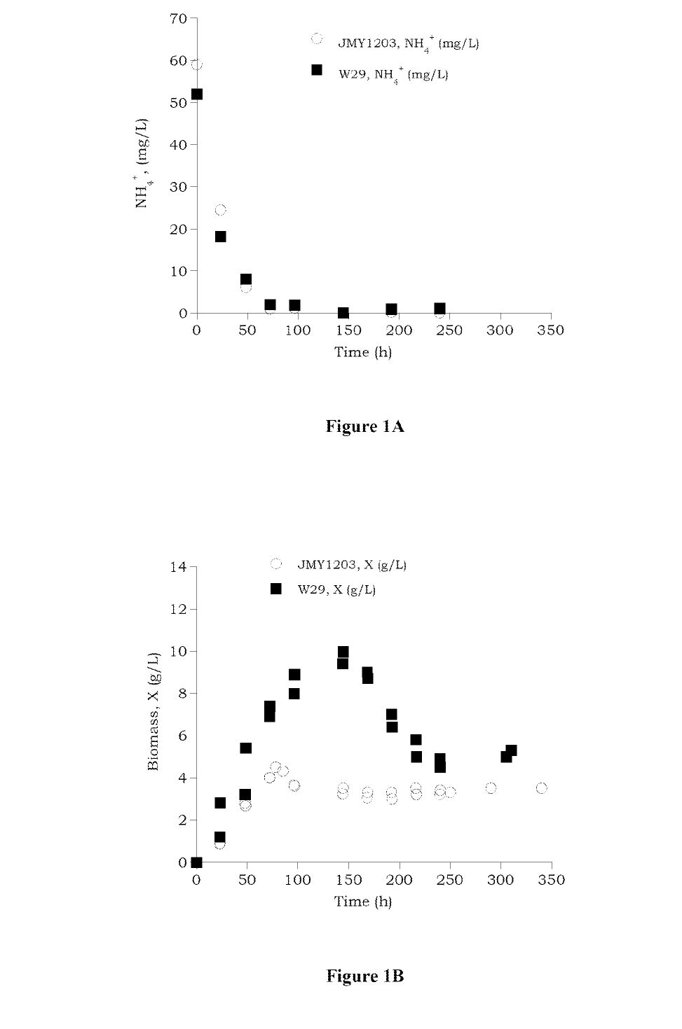

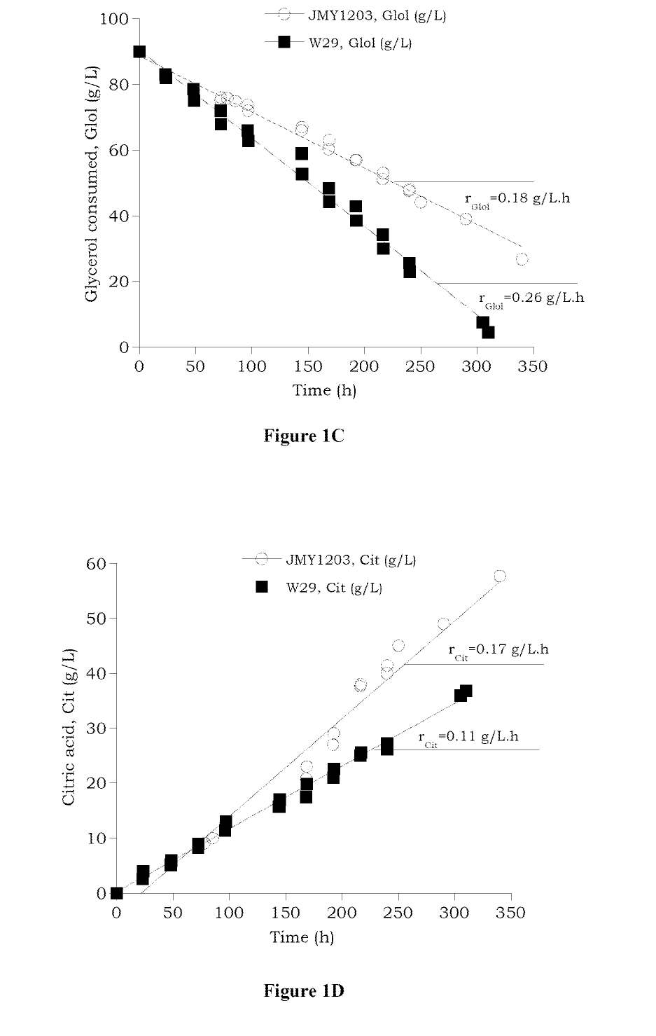

FIG. 1: Time course of ammonium ion consumption (A), of biomass production (B), of glycerol consumption (C) and of total citric acid production (D) during the growth of the Yarrowia lipolytica strains W29 and JMY1203 cultured on a nitrogen-limited glycerol-based (Glol) medium. Culture conditions: growth in 250 ml flasks at 185 rpm, Glol₀=90 g/l, initial pH=6.0±0.1 then maintained between 4.8 and 6.0, DOT>40% (v/v), incubation temperature of 28° C. Each point represents the mean of two independent measurements.

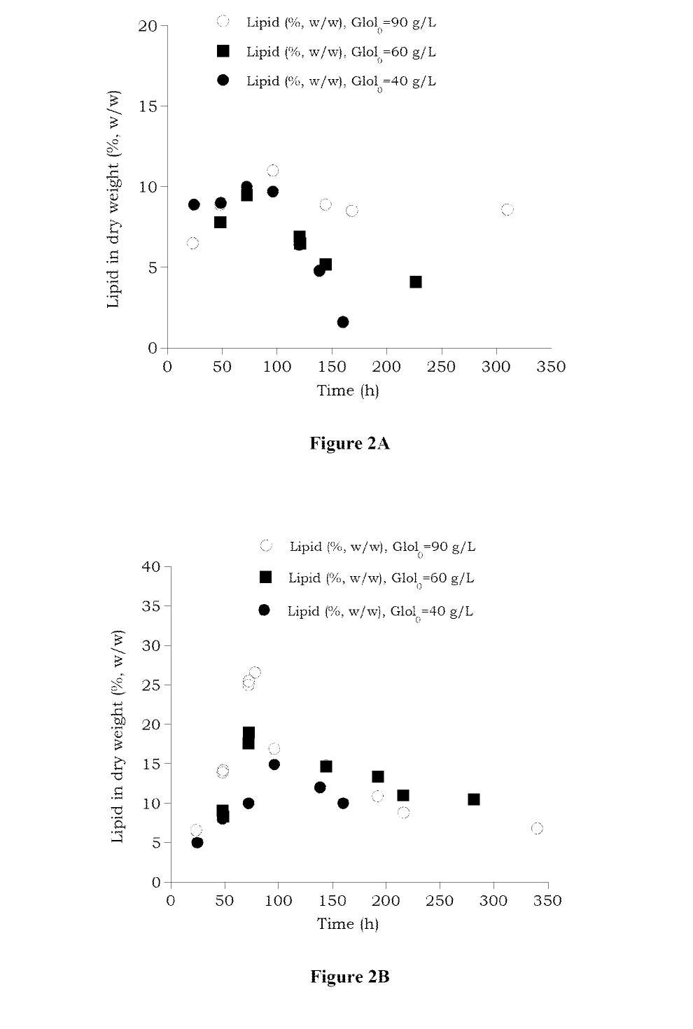

FIG. 2: Time course of total lipids in the dry biomass (%, w/w) during the growth of the Yarrowia lipolytica strains W29 (A) and JMY1203 (B) in a nitrogen-limited, glycerol-based medium. Culture conditions: growth in 250 ml flasks at 185 rpm, Glol₀=40, 60 and 90 g/l, initial pH=6.0±0.1 then maintained between 4.8 and 6.0, DOT>40% (v/v), incubation temperature of 28° C. Each point represents the mean of two independent measurements.

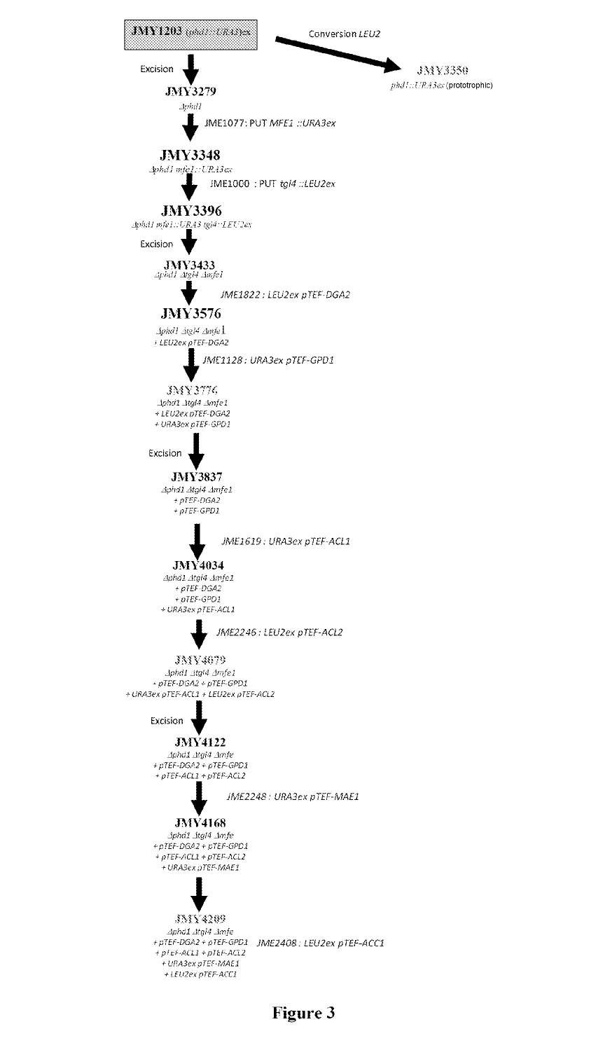

FIG. 3: Diagrammatic representation of the construction of mutant strains according to the invention.

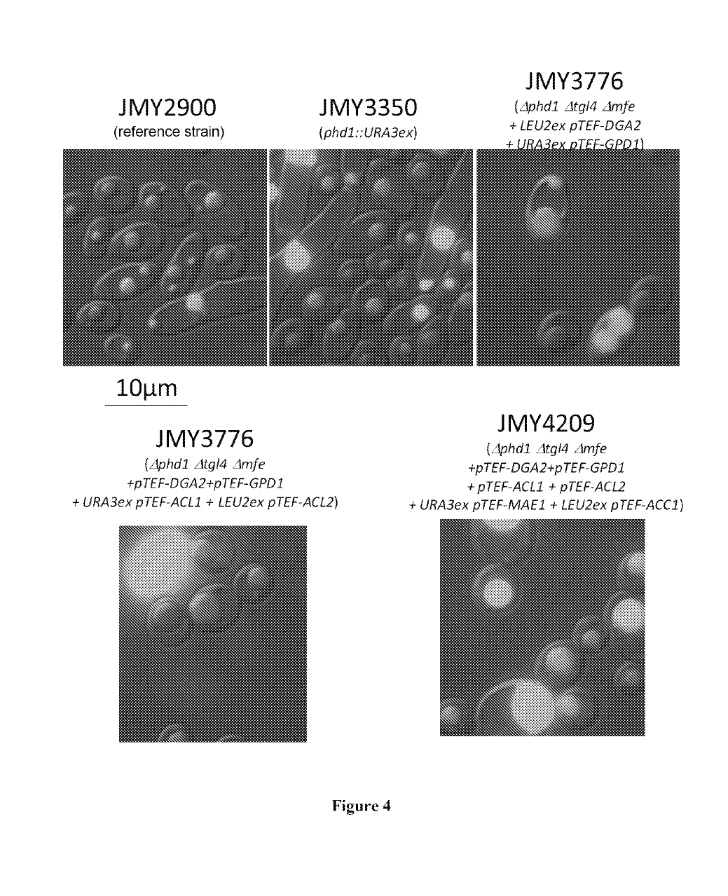

FIG. 4: Visualization of lipid accumulation by BodiPy staining of the lipid bodies produced by the JMY3776 and JMY4209 strains.

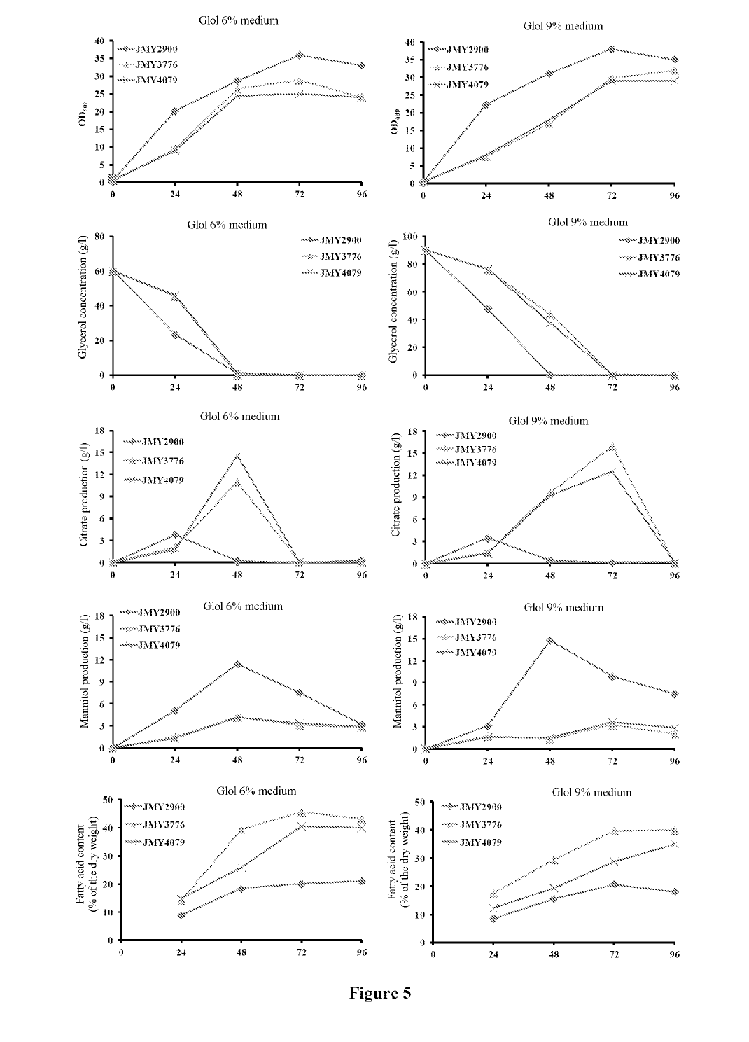

FIG. 5: Monitoring of various parameters (growth, glycerol consumption, citrate, mannitol and fatty acid production) during the growth of the JMY2900, JMY3776 and JMY4079 strains, in the Glol 6% and Glol 9% media.

### EXAMPLE

### Obtaining and Characterization of Mutant Yarrowia Lipolytica Yeast Strains in which at Least the Expression of 2-Methylcitrate Dehydratase is Inhibited

### 1) Materials and Methods

i) Strains and Media

The mutant Y. lipolytica strains according to the present invention are derived from the auxotrophic Y. lipolytica strain Pold (Leu⁻ Ura⁻; CUB 139; of genotype MatA Ura3-302, Leu2-270, xpr2-322), itself derived from the wild-type Y. lipolytica strain W29 (of genotype MatA; ATCC20460) by genetic modification. The Pold and W29 strains were described by Barth and Gaillardin (1996). These two strains Pold and W29 do not exhibit any differences with regard to the production of lipids and of citric acid. The yeast cells were cultured on YPD medium (Barth et al., 1996) or YNBCas medium (YNBD with 0.2% casamino acids) for the selection of the transformants.

The Escherichia coli strain Mach1-T1 (Invitrogen) was used for the transformation and amplification of the recombinant plasmid DNA. The cells were cultured on an LB medium (Sambrook et al., 1989). Kanamycin (40 μg/ml) was used for the plasmid selection.

ii) Construction of the YALI0F02497 Deletion Cassette

The PHD1 gene (YALI0F02497) of the Y. lipolytica strain Po1d was deleted by replacing the coding region of this gene with a cassette containing the URA3 gene as selectable marker, according to the gene disruption method described by Fickers et al. (2003). More specifically, the promoter (P) and terminator (T) regions of the YALI0F02497 gene [T1] were obtained by PCR amplification of the genomic DNA of Y. lipolytica W29 using the pairs of primers YALI0F02497-P1 (SEQ ID NO: 2)/YALI0F02497-P2 (SEQ ID NO: 3) to amplify the promoter region, and YALI0F02497-T1 (SEQ ID NO: 4)/YALI0F02497-T2 (SEQ ID NO: 5) to amplify the terminator region. The YALI0F02497-P2 and YALI0F02497-T1 primers were designed to introduce an IsceI restriction site at the 3′ end of the P fragment and at the 5′ end of the T fragment. The corresponding P-IsceI and T-IsceI fragments were grouped together and used as templates for the amplification of the P-IsceI-T cassette with the pair of primers YALI0F02497-P1/YALI0F02497-T2. The P-IsceI-T cassette was cloned into the pCR4® Blunt-TOPO plasmid (Invitrogen, Cergy-Pontoise, France), and used to transform the E. coli strain Mach1-T1 (Invitrogen). The resulting construct, called pYALI0F02497-PT (JME739), was verified by restriction analysis with IsceI and sequenced. The loxR-URA3-loxP fragment encoding the URA3 gene was excised from the JMP121 plasmid (Fickers et al., 2003) by IsceI restriction and cloned at the corresponding site in pYALI0F02497-PT so as to insert the URA3 selectable marker between the P and T fragment of the P-IsceI-T cassette at the level of the IsceI site. The resulting construct, called pYALI0F02497-PUT (JME740) comprises the PUT cassette of the YALI0F02497 gene (YALI0F02497-PUT cassette).

The ΔYALI0F02497::URA3 deletion was introduced into the Y. lipolytica strain Po1d (JMY195), according to the method described by Fickers et al. (2003), giving rise to the deleted strain JMY1203 (of genotype MatA, Ura3-302, Leu2-270, xpr2-322, ΔYALI0F02497:: URA3). The disruption cassette was amplified by PCR and used to transform the Y. lipolytica strain Po1d. The Ura⁺ transformants were selected on YNBCas medium. The disruption of the gene was verified by PCR using the pair of primers YALI0F02497-ver1 (SEQ ID NO: 6)/YALI0F02497-ver2 (SEQ ID NO: 7). Two transformants (YALI0F02497-1 and YALI0F02497-5) exhibited a PCR fragment of 3.7 kb corresponding to the disrupted gene. The disruption of the gene in these two transformants was confirmed by Southern blotting.

iii) Construction of the Vectors for Overexpression of the ACL1 and ACL2 Genes, of the MAE1 Gene and of the ACC1 Gene

The JME1619 and JME2246 vectors were constructed by cloning the coding sequences of the ACL1 and ACL2 genes between the BamHI and AvrII restriction sites of the JMP62-pTEF-URA3ex (Beopoulos et al., 2012) and JMP62-pTEF-LEU2ex (Beopoulos et al., 2014) vectors, respectively. For that, the coding sequences of the ACL1 and ACL2 genes were amplified using the following oligonucleotides:

For ACL1:

ACL1-S:
(SEQ ID NO: 9)
CGCGGATCCCACAATGTCTGCCAACGAGAACATCTCCCGATTCGAC,

sense oligonucleotide, bearing the BamHI restriction site.

ACL1-A:
(SEQ ID NO: 10)
CACCCTAGGTCTATGATCGAGTCTTGGCCTTGGAAACGTC,

antisense oligonucleotide, bearing the AvrII restriction site.

The resulting amplicon was then digested with the BamHI and AvrII enzymes and cloned into the JMP62-pTEF-LEU2ex vector, generating the JME1619 vector.

For ACL2:

The coding sequence of ACL2 contains two BamHI restriction sites; the cloning of this sequence required the use of various oligonucleotides in order to delete these restriction sites, without however modifying the sequence of the protein derived from this gene.

ACL2-A:
(SEQ ID NO: 11)
CACGGATCCCACAATGTCAGCGAAATCCATTCACGAGGCCGAC,

sense oligonucleotide, bearing the BamHI restriction site.

ACL2-B:
(SEQ ID NO: 12)
ATGCCTAGGTTAAACTCCGAGAGGAGTGGAAGCCTCAGTAGAAG,

antisense oligonucleotide, bearing the AvrII restriction site.

| (SEQ ID NO: 13) | |
|---|---|
| ACL2-C: | GAGAGGGCGACTGGAT [symbol] CTCTTCTACCAC. |

sense oligonucleotide, bearing a mutation which makes it possible to delete a BamHI restriction site.

| (SEQ ID NO: 14) | |
|---|---|
| ACL2-Dd: | GTGGTAGAAGAGAATC [symbol] AGTCGCCCTCTC, |

antisense oligonucleotide, bearing a mutation which makes it possible to delete a BamHI restriction site.

| (SEQ ID NO: 15) | |
|---|---|
| ACL2-E: | CTTCACCCAGGTTGG [symbol] TCCACCTTCAAGGGC, |

sense oligonucleotide, bearing a mutation which makes it possible to delete a BamHI restriction site.

| (SEQ ID NO: 16) | |
|---|---|
| ACL2-F: | GCCCTTGAAGGTGGA [symbol] CCAACCTGGGTGAAG, |

antisense oligonucleotide, bearing a mutation which makes it possible to delete a BamHI restriction site.

In order to reconstitute an ACL2 gene compatible for cloning into the BamHI and AvrII sites of the JMP62-pTEF-LEU2ex vector, three amplicons using the ACL2-A/ACL2-Dd, ACL2-C/ACL2-F and ACL2-B/ACL2-E primers were amplified. Finally, these amplicons were joined together end to end by fusion PCR using the ACL2-A and ACL2-B oligonucleotides. The resulting amplicon was then digested with the BamHI and AvrII enzymes and cloned into the JMP62-pTEF-LEU2ex vector, generating the JME2246 vector.

For MAE1:

| (SEQ ID NO: 17) | |
|---|---|
| MAE1-sense: | CGCGGATCCCACAATGTTACGAC, |

sense oligonucleotide, bearing the BamHI restriction site.

| (SEQ ID NO: 18) | |
|---|---|
| ACL1-antisense: | GCGCCTAGGCTAGTCGTAATCCCG, |

antisense oligonucleotide, bearing the AvrII restriction site.

The resulting amplicon was then digested with the BamHI and AvrII enzymes and cloned into the JMP62-pTEF-URA3ex vector, generating the JME2248 vector.

For ACC1:

BamCytoATG:
(SEQ ID NO: 19)
AACGCGGATCCCACAATGGCTTCAGGATCTTCAACG,

sense oligonucleotide, bearing the BamHI restriction site.

ACCavSph:
(SEQ ID NO: 20)
GTCCAAGCTCGGGAAGCTG
ACCrevIntron:
(SEQ ID NO: 21)
CCGTTGTTAGCGATGAGGACCTTGTTGATAACTGTATGACCTC
ACCdirIntron:
(SEQ ID NO: 22)
GAGGTCATACAGTTATCAACAAGGTCCTCATCGCTAACAACG
ACCamXba:
(SEQ ID NO: 23)
AGTATCTCATTTCCGAGGCTG
ACCdirBamKO:
(SEQ ID NO: 24)
CTGGACACCATGGCTCGTCTTGATCCCGAGTACTCCTCTCTC
ACCrevBamKO:
(SEQ ID NO: 24)
GAGAGAGGAGTACTCGGGATCAAGACGAGCCATGGTGTCCAG
AvrRevACC:
(SEQ ID NO: 26)
AGCTATCGATAATCCTAGGTCACAACCCCTTGAGCAGCTC,

antisense oligonucleotide, bearing the ClaI and AvrII sites.

Since the ACC1 gene is particularly long and contains an intron (containing a NotI site, which is important in the release of the overexpression cassette), and restriction sites (NotI in the intron and a BamHI site) which are unsuitable for cloning into the JMP62-pTEF-LEU2ex vector opened with BamHI and AvrII, various amplicons were amplified in order to delete the intron and the NotI and BamHI restriction sites. Thus, 4 amplicons were obtained using the following pairs of primers:

Amplicon 1: BamCytoATG and ACCrevIntron (184 bp),

Amplicon 2: ACCdirIntron and ACCavSph (2207 bp),

Amplicon 3: ACCamXba and ACCrevBamKO (2101 bp),

Amplicon 4: ACCdirBamKO and AvrRevACC (585 bp),

Amplicon 5: BamCytoATG and AvrRevACC (7270 bp).

Amplicons 1 and 2 were then fused with the BamCytoATG and ACCavSph primers. Amplicons 1+2 and amplicon 5 were digested with BamHI+SphI. Amplicon 5, digested with BamHI+SphI, makes it possible to obtain a 1876 bp fragment, called fragment 5′. The fragments thus digested (1+2 and 5′) were subsequently cloned by 3-way ligation between the BamHI and XbaI sites of the Bluescript(−)KS vector, generating the JME2412 vector. Amplicons 3 and 4 were then fused with the ACCamXba and AvrRevACC primers. Amplicon 3+4 was then digested with XbaI and ClaI and cloned by 3-way ligation between the XbaI and ClaI sites of the Bluescript(−)KS vector, generating the JME2413 vector. The JME2412 and JME2413 vectors were then digested with XbaI and ClaI in order to release the fragments 1+2+5′ and 3+4 with compatible ends. These two fragments thus digested were subsequently cloned by 3-way ligation between the BamHI and ClaI sites of the Bluescript(−)KS vector, generating the JME2406 vector. The coding sequence of the ACC1 gene thus reconstructed was finally digested with the BamHI+AvrII enzymes, in order to be cloned between the BamHI+AvrII sites of the JMP62 pTEF-LEU2ex vector, generating the JME2408 vector.

iv) Construction of the Mutant Strains Derived from the Δphd1(JMY1203) Strain

The JMY1203 strain was rendered protrophic by conversion of the leu2-270 locus into its wild-type version. The JMY3279 strain was obtained after excision of the URA3ex selectable marker from the JMY1203 strain, according to the principle described by Fickers et al. (2003). This strain was then successively transformed with the cassettes for disruption of the MFE1 (JME1077) and TGL4 (JME1000) genes, already described in Dulermo and Nicaud (2011) and Dulermo et al. (2013), respectively. The URA3ex and LEU2ex markers of the JMY3396 strain thus obtained were then excised (Fickers et al., 2003), generating the JMY3433 strain. The latter was then successively transformed with the LEU2ex pTEF-DGA2 (JME1822, NotI digestion, derived from JME1132, Beopoulos et al., 2012) and URA3ex pTEF-GPD1 (JME1128, NotI digestion, Dulermo and Nicaud, 2011) overexpression cassettes, generating the JMY3776 strain. The JMY4079 strain was obtained after excision of the URA3ex and LEU2ex selectable markers (Fickers et al., 2003), then successive transformation with the cassettes for overexpression of the ACL1 (JME1619) and ACL2 (JME2246) genes. The URA3ex and LEU2ex markers of the JMY4079 strain were then excised (Fickers et al., 2003), generating the JMY4122 strain. The latter was then successively transformed with the URA3ex pTEF-MAE1 (JME2248, NotI digestion) and LEU2ex pTEF-ACC1 (JME2408, NotI digestion) overexpression cassettes, thus generating the JMY4168 and JMY4209 strains, respectively.

A diagram representing the construction of the various strains and also the vectors used is given in FIG. 3.

v) Culture Conditions for the W29 and JMY1203 Strains

The wild-type Y. lipolytica strain W29 and the genetically modified strains were used for the fermentations.

All the experiments were carried out in culture flasks with shaking. The culture medium used contained (in g/l): KH₂PO₄ 7.0; Na₂HPO₄ 2.5; MgSO₄×7H₂O 1.5; CaCl₂×2H₂O 0.1; FeCl₃×6H₂O 0.15; ZnSO₄×7H₂O 0.02; MnSO₄×H₂O 0.06 (Papanikolaou et al., 2002). Ammonium sulfate and yeast extract were used as nitrogen sources at a concentration of from 0.25 to 2.5 g/l respectively. Crude glycerol (Industrie Hellénique de la Glycerine et des Acides Gras SA; purity approximately 70%, g/g, impurities composed of potassium and sodium salts 12%, w/w, of non-glycerol organic material 1%, v/v, of water 17%, g/g and of methanol<0.1%, g/g) was used as sole carbon source at different concentrations. The initial pH for all the media is 6.0±0.1. For the controlled experiments, glucose of analytical quality (AnalaR, BDH, United Kingdom) was used as carbon source.

250 ml conical flasks filled with 50±1 ml of culture medium were inoculated with 1 ml of preculture in the exponential growth phase, containing 1-3×10⁶ cells (concentration of the initial biomass X₀ approximately 0.10 g/l). The flasks were incubated at a temperature of 28° C. and shaken at 180 rpm in a rotary shaker (New Brunswick Sc, United States). The preculture was carried out in the synthetic medium mentioned above with pure glycerol (purity 98%) used as substrate at 20 g/l.

vi) Culture Conditions for the JMY2900, JMY3776 and JMY4079 Strains

The Y. lipolytica strains JMY2900 (reference), JMY3776 (Δphd1 Δmfe1 Δtgl4+pTEF-DGA2-LEU2ex+pTEF-GPD1-URA3ex) and the strain JMY4079 (Δphd1 Δmfe1 Δtgl4+pTEF-DGA2+pTEF-GPD1+pTEF-ACL1-URA3ex+pTEF-LEU2ex) were evaluated for their capacity to produce lipids, in baffled flasks.

The culture medium used contained: 60 g/l of pure glycerol (Glol 6% medium) or 90 g/l of pure glycerol (Glol 9% medium), 5 g/l of NH₄Cl as sole nitrogen source and 1.7 g/l of YNB. A 50 mM phosphate buffer (35 mM KH₂PO₄, 64 mM Na₂HPO₄) is added in order to maintain the pH of the medium at 6.8±0.1.

500 ml conical flasks filled with 50±1 ml of culture medium were inoculated with 1 ml of preculture in the exponential growth phase, containing 1-3×10⁶ cells (concentration of the initial biomass X₀ approximately 0.10 g/l). The flasks were incubated at a temperature of 28° C. and shaken at 160 rpm in a rotary shaker (New Brunswick Sc, United States). The preculture was carried out in the synthetic media mentioned above.

vii) Analytical Methods

In all the tests, the production of dry biomass, the consumption of glucose or of glycerol, the secretion of organic acids, the concentration of residual nitrogen and the intracellular production of lipids were evaluated. The initial pH of the culture medium was 6.0±0.1. During the cultures, the pH was maintained within a range of between 4.8 and 6.0 by periodic addition of 5M KOH. The dissolved oxygen tension (% DOT, v/v) was measured using a selective electrode (Sensodirect Oxi 200, Lovibond). All the tests were carried out under aerobic conditions (DOT>40%, v/v, for all the growth phases). The yeast cells were harvested by centrifugation (Hettich Universal 320-R, Germany) at 10 000×g/15 min and washed 3 times with distilled water. The concentration of biomass (X, g/l) was determined by the dry weight (85±5° C./24 h). The glycerol (Glol, in g/l), the glucose (Glc, in g/l) and the organic acids were analyzed by HPLC as described by André et al. (2009). The concentration of isocitric acid was determined by means of an enzymatic process, by measuring the NADPH₂ produced during the conversion of the isocitric acid into α-ketoglutaric acid, catalyzed by isocitrate dehydrogenase, as described by Papanikolaou et al. (2002). The total amount of citric acid (citric and isocitric acid) produced was characterized as Cit (in g/l). The ammonium ion determination was carried out using an ammonium selective electrode (Hach 95-12, Germany).

The total cellular lipids (L, in g/l) were extracted from the dry biomass with a 2/1 (v/v) chloroform/methanol mixture and were determined by gravimetric analysis. The cellular lipids were fractionated into their lipid fractions. Briefly, a known weight of extracted lipids (approximately 200 mg) was dissolved in chloroform (3 ml) and was fractionated using a column (25×100 mm) of silicic acid, activated by heating overnight at 110° C. (Fakas et al., 2006). Successive applications of chloroform, of acetone and of methanol produce fractions containing neutral lipids (N), glycolipids plus sphingolipids (G+S) and phospholipids (P), respectively (Guo et al., 2000; Fakas et al., 2006). The weight of each fraction was determined after evaporation of the respective solvent. The total cellular lipids or the individual lipid fractions were converted into their fatty acid methyl esters (FAMEs) during a two-step reaction with methanolic sodium and methanolic hydrochloric acid (Fakas et al., 2006). This method was chosen so as to avoid trans-isomerization of the fatty acids. The FAMEs were analyzed in a gas chromatography apparatus (GC-FID) (Fisons series 8000) according to Fakas et al. (2006). The FAMEs were identified by comparison with standards.

viii) Analytical Methods for the JMY2900, JMY3776 and JMY4079 Strains

In all the tests, the production of dry biomass, the consumption of glycerol, the secretion of organic acids and the intracellular production of lipids were evaluated. The yeast cells were harvested by centrifugation (Hettich Universal 320-R, Germany) at 4000×g/5 min and washed 3 times with distilled water. The concentration of biomass (X, g/l) was determined by the dry weight (lyophilization for 48 h). The glycerol (Glol, in g/l), and the organic acids were analyzed by HPLC as described by Lazar et al. (2013). The amount of citric acid produced was characterized as CA (in g/l).

The total cellular lipids (L, in g/l) were extracted from the ground dry biomass (20 to 30 mg) with a 2/1 (v/v) chloroform/methanol mixture, according to the protocol of Folch and Lee (1957) and were determined by gravimetric analysis. The total cellular lipids were converted into their fatty acid methyl esters (FAMEs) by the method of Browse (Browse et al., 1986). The FAMEs were analyzed in a gas chromatography apparatus (GC-FID) (Varian, GC-430) according to Beopoulos et al. (2008). The FAMEs were identified by comparison with standards.

### 2) Results

i) Growth of the Y. lipolytica Strains on Glucose or Crude Glycerol

The Y. lipolytica strains W29 and JMY1203 were cultured in a nitrogen-limited (deficient) medium with an initial concentration of glycerol (Glol0) or of glucose (Glc0) adjusted to 40 g/l. The cultures of these two strains on glucose are considered to be a basis for comparison. The results of the time course are described in table 2 hereinafter.

TABLE 2
Quantitative data for the Y. lipolytica strains W29 and JMY1203 originating from cultures
on nitrogen-limited medium containing glucose or glycerol as substrate at an initial
concentration of 40 g/l. Representation of the biomass (X, g/l), of the lipids (L, g/l),
of the total citric acid (Cit, g/l), of the glycerol consumed (Glol_{cons}, g/l) and of the
glucose consumed (Glc_{cons}, g/l) when: the maximum amount of lipids in the dry weight
of yeast (%, g/g) (a) and the maximum concentration of total citric acid (b) are reached.
Culture conditions: growth in 250 ml flasks at 185 rpm, initial pH = 6.0 ± 0.1,
then maintained between 4.8 and 6.0, DOT > 40% (v/v), incubation temperature of
28° C. Each point represents the mean of two independent measurements.

| | | | | | | | | Y_{L/Glol} | | Y_{Cit/Glol} |
|---|---|---|---|---|---|---|---|---|---|---|
| | Carbon | Time | Glol_{cons} | Glc_{cons} | X | Lipids | L | (Y_{L/Glc}) | Cit | (Y_{Cit/Glc}) |
| Strain | source | (h) | (g/l) | (g/l) | (g/l) | (%, g/g) | (g/l) | (g/g) | (g/l) | (g/g) |
| W29 | Glc a | 91.5 | — | 21.9 | 11.2 | 5.8 | 0.65 | 0.030 | 18.2 | 0.83 |
| | Glc b | 170 | — | 37.0 | 11.0 | 2.4 | 0.26 | 0.007 | 31.4 | 0.85 |
| JMY1203 | Glc a | 72.5 | — | 12.2 | 3.2 | 10.1 | 0.32 | 0.026 | 6.8 | 0.56 |
| | Glc b | 150 | — | 34.5 | 1.8 | 5.1 | 0.09 | 0.003 | 15.2 | 0.44 |
| W29 | Glol a | 72 | 12.9 | — | 10.7 | 10.0 | 1.07 | 0.083 | 6.0 | 0.47 |
| | Glol b | 160 | 39.9 | — | 12.5 | 1.6 | 0.20 | 0.005 | 19.1 | 0.48 |
| JMY1203 | Glol a | 96 | 19.0 | — | 5.5 | 14.9 | 0.82 | 0.043 | 11.8 | 0.62 |
| | Glol b | 160 | 39.9 | — | 7.0 | 10.0 | 0.70 | 0.018 | 31.0 | 0.78 |

In all the experiments and independently of the carbon source used, the W29 and JMY1203 strains consumed, with comparable rates, the available extracellular nitrogen (initial NH4⁺ at 55±10 ppm, exhaustion of the nitrogen in 60±5 hours after inoculation). The W29 strain exhibits a biomass production which is greater than that of the JMY1203 strain on the two substrates, of between 10.7-12.5 g/l. For the JMY1203 strain, the biomass production reaches a maximum of 7 g/l in the presence of glycerol; on glucose, the biomass concentration decreases during the culture down to 1.8 g/l, suggesting cell lysis at the end of culture.

The citrate production increases after exhaustion of the nitrogen in the medium, resulting in its secretion. For the W29 strain, the citrate production is greater on glucose, reaching Citₘₐₓ=31.5 g/l with a degree of conversion of Y_{Cit/Glc}=0.85 g/g. On glycerol, the maximum citrate production is 1.65 times lower (Citₘₐₓ=19.1 g/l, degree of glycerol bioconversion=0.48 g/g) than on glucose medium and the degree of conversion decreased by 56%. The JM1203 strain exhibits opposite characteristics on glucose; the citrate production and the degrees of conversion are Citₘₐₓ=15.2 g/l and Y_{Cit/Glol}=0.44 g/g, respectively, whereas on crude glycerol the Citₘₐₓ was 2.04 times higher (31 g/l), corresponding to a 37.4% increase in the degree of conversion reaching 0.78 g/g. These results indicate a difference in carbon flow according to growth on glucose or glycerol for the two strains, which exhibit an opposite phenotype.

Furthermore, even though the JMY1203 strain produces less biomass compared with the W29 strain, it exhibits an increase of 1.74 times more lipids, reaching 10.1%, g/g, of the dry weight (DW), on glucose and 1.49 times more lipids on glycerol, reaching 14.9%, g/g, of the DW. Glycerol is a better substrate with regard to lipid accumulation for these two strains.

A rapid decrease in the accumulated lipids is observed for the W29 strain, where the lipid content decreases from 5.8% to 2.4%, g/g, of the DW (58% decrease in the lipid content) on glucose and from 10% to 1.6%, g/g, of the DW on glycerol. This demonstrates a significant remobilization of the accumulated lipids with a simultaneous increase in citric acid production, particularly in the presence of glycerol. For the JMY1203 strain, the amount of accumulated lipids decreases from 10.1% to 5.1%, g/g, of the DW (49.5% decrease) on glucose and from 14.9% to 10%, g/g, of the DW (32.9% decrease) on glycerol. It is therefore observed that the W29 strain remobilizes these lipid stores on glucose more rapidly than the JMY1203 strain. On glycerol medium, the remobilization is similar for the two strains.
ii) Growth of the Y. lipolytica Strains on Crude Glycerol at High Initial Substrate Concentrations

Nitrogen-limited media containing the same amount of initial nitrogen as that previously described, but a higher initial concentration of crude glycerol, were used (Glol₀ at 60 and 90 g/l). The results of the time course are described in table 3 hereinafter.

TABLE 3
Quantitative data for the Y. lipolytica strains W29 and JMY1203 originating from
cultures on medium which is nitrogen-limited at a constant initial concentration
and contains glycerol as substrate at two differential initial concentrations
(Glol₀, g/l). Representation of the biomass (X, g/l), of the lipids (L, g/l), of
the total citric acid (Cit, g/l) and of the glycerol consumed (Glol_{cons}, g/l)
when: the maximum amount of lipids in the dry weight of yeast (%, g/g) (a) and
the maximum concentration of total citric acid (b) are reached. Culture conditions:
growth in 250 ml flasks at 185 rpm, Glol₀ = 60 or 90 g/l, initial pH = 6.0 ± 0.1,
then maintained between 4.8 and 6.0, DOT > 40% (v/v), incubation temperature
of 28° C. Each point represents the mean of two independent measurements.

| | Glol₀ | Time | Glol_{cons} | X | Lipids | L | Y_{L/Glol} | Cit | Y_{Cit/Glol} |
|---|---|---|---|---|---|---|---|---|---|
| Strain | (g/l) | (h) | (g/l) | (g/l) | (%, g/g) | (g/l) | (g/g) | (g/l) | (g/g) |
| W29 | 60 a | 72 | 19.7 | 7.6 | 9.5 | 0.73 | 0.037 | 1.9 | 0.10 |
| | 60 b | 226 | 59.9 | 10.7 | 4.1 | 0.44 | 0.007 | 27.0 | 0.45 |
| JMY1203 | 60 a | 72.5 | 16.0 | 3.1 | 19.0 | 0.59 | 0.031 | 4.1 | 0.26 |
| | 60 b | 281 | 60.1 | 3.9 | 10.5 | 0.41 | 0.007 | 45.5 | 0.76 |
| W29 | 90 a | 96 | 24.0 | 8.0 | 11.1 | 0.88 | 0.037 | 11.4 | 0.47 |
| | 90 b | 310 | 85.5 | 5.3 | 8.6 | 0.46 | 0.005 | 36.8 | 0.43 |
| JMY1203 | 90 a | 78 | 14.4 | 4.5 | 26.6 | 1.20 | 0.083 | 9.1 | 0.64 |
| | 90 b | 340 | 63.2 | 3.5 | 6.9 | 0.24 | 0.004 | 57.7 | 0.91 |

The graphic representation of the time courses for the W29 and JMY1203 strains during the Glol₀=90 g/l test is shown in FIG. 1. The extracellular nitrogen (initial NH4⁺ at 55±10 mg/1) was exhausted at approximately 60 h after inoculation for the two strains (FIG. 1A). The degree of nitrogen absorption was similar independently of the Glol₀ concentration employed or of the use of glucose as substrate, for the two strains tested (time courses not shown). Despite the nitrogen exhaustion, the concentration of the biomass clearly increased for the W29 strain, reaching the value Xₘₐₓ of approximately 10 g/l after 150 h (FIG. 1B) and then rapidly decreased. The JMY1203 strain exhibits a lower growth rate; the production of biomass stopped after the exhaustion of the assimilable nitrogen in the culture medium and was kept constant until the end of the culture.

The consumption of glycerol was constant and virtually linear for the two strains tested, both in the equilibrated growth phase in nitrogen (0-60 h) and the limited growth phase (60 to 330 h), with a glycerol consumption level r_{Glol} (=−ΔGol/Δt) of 0.26 g/l for the W29 strain, a value clearly higher than 0.18 g/l obtained for the JMY1203 strain (FIG. 1C). Although citric acid was produced by the two strains in the equilibrated growth phase, the Cit production mainly occurred after nitrogen exhaustion of the medium. The citric acid production appears to be almost linear for the W29 strain with a level of total citric acid production r_{Cit} (=ΔCit/Δt) of 0.11 g/l, whereas this value is 1.54 times higher for the JMY1203 strain (0.17 g/l·h; FIG. 1D). The two strains exhibit the same behavior regardless of the glycerol concentration, thereby indicating that the levels of glycerol consumption and of citrate production are strain-dependent (wild-type vs mutant).

With regard to the lipid accumulation, the two strains exhibited completely different behavior (FIG. 2A, B). During the phase which is not nitrogen-limiting and regardless of the glycerol concentration, the W29 strain accumulated lipids with a similar level, to reach a maximum of 10% g/g of the DW under all the conditions tested. However, after exhaustion of the nitrogen, the lipids are rapidly remobilized at low glycerol concentrations, whereas, at high glycerol concentrations, the lipids are not remobilized (FIG. 3A). This behavior suggests a regulation of the lipid degradation pathway as a function of the glycerol concentration. On the other hand, for the JMY1203 strain, the lipid accumulation clearly depends on the glycerol concentration during the growth phase where nitrogen was not limiting, while the degradation of the lipids during the nitrogen-deficient phase was not affected as a function of the glycerol concentration (FIG. 2B). The lipid accumulation reaches 26.6%, g/g, of the DW for an initial glycerol concentration (Glol₀) of 90 g/l, and 14.9% for a Glol₀ of 40 g/l.

The W29 strain exhibited a higher concentration of the biomass compared with the JMY1203 strain. As previously, the increase in the Glol₀ concentration gave rise to a decrease in the amount of biomass (X) produced by the W29 strain, suggesting a potential inhibition of the substrate. A similar observation can also be made for the JMY1203 strain, taking into account the fact that the amount X is approximately 7 g/l for the test with Glol₀=40 g/l, and approximately 4 g/l for the test with Glol₀=90 g/l.

Furthermore, the Cit concentration (in g/l) substantially increased with the increase in the Glol₀ concentration of the medium; the W29 strain produces twice as much citrate at Glol₀=90 g/l, compared with the amount of citrate produced at Glol₀=40 g/l. However, the conversion yield for the citric acid produced per glycerol consumed (Y_{Cit/Glol}) remained relatively constant at 0.45 g/g (see tables 3 above and 4 below), reaching a maximum of 0.48 g/g, thereby suggesting that this is the threshold of bioconversion of glycerol to citric acid for the W29 strain, under these culture conditions. For the JMY1203 strain, even though the biomass obtained was much smaller compared with the W29 strain, the citrate production and the degree of glycerol bioconversion increase with the glycerol concentration. The amount of citrate went from 31 g/l to approximately 58 g/l and the yield of bioconversion of glycerol to citric acid (Y_{Cit/Glol} value) went from 0.78 g/g at Glol₀=40 g/l to the impressive value of 0.91 g/g at Glol₀=90 g/l. These values represent an increase of 1.6-fold for the citrate production and of 2.1-fold for the yield of conversion of glycerol to citrate, compared with the W29 strain. Citric acid is the principal compound of the total citrate produced, since quantitative determination of the isocitric acid showed that the isocitric acid was approximately 5-8%, g/g, of the total citric acid produced, whatever the strain tested and the Glol₀ concentration of the medium. In the test with the Citₘₐₓ amount reached, the amount of isocitric acid quantitatively determined was approximately 5%, g/g of the Cit.

The exceptional Y_{Cit/Glol} value that was obtained shows that the genetically modified JMY1203 strain can be used to promote the conversion of crude glycerol to citric acid.

iii) Lipid Analysis

The fatty acid (FA) composition of the cellular lipids produced was studied at the end of the growth phases for the two strains cultured on glucose and crude glycerol. It is represented in tables 4 (W29 strain) and 5 (JMY1203 strain) hereinafter.

TABLE 4
Fatty acid composition of the total cellular lipids produced by the Yarrowia lipolytica
strain W29 during its growth in a nitrogen-limited medium containing glucose
(at 40 g/l) or glycerol (at 40, 60 or 90 g/l). The culture conditions are
identical to those described for tables 2 and 3.

| | | Fatty acids (%, w/w) | | | | |
|---|---|---|---|---|---|---|
| Substrate | Growth phase | C16:0 | ^{Δ9}C16:1 | C18:0 | ^{Δ9}C18:1 | ^{Δ9,12}C18:2 |
| Glc₀ = 40 g/l | LE or ES (60-90) | 12.7 | 6.2 | 5.3 | 55.1 | 20.4 |
| | S (110-160) | 19.2 | 6.9 | 8.1 | 44.6 | 20.9 |
| Glol₀ = 40 g/l | LE or ES (60-90) | 17.8 | 9.6 | 15.1 | 54.2 | 2.8 |
| | S (110-160) | 22.3 | 14.1 | 9.0 | 48.1 | 6.2 |
| Glol₀ = 60 g/l | LE or ES (60-90) | 21.5 | 6.9 | 18.1 | 44.9 | 9.9 |
| | S (110-160) | 20.1 | 8.4 | 19.5 | 40.8 | 11.2 |
| Glol₀ = 90 g/l | LE or ES (60-90) | 15.9 | 6.0 | 15.5 | 47.1 | 9.1 |
| | S (110-160) | 16.8 | 6.8 | 14.5 | 46.7 | 14.9 |

LE: end of the exponential phase.
ES: start of the stationary phase.
S: stationary phase.

TABLE 5
Fatty acid composition of the total cellular lipids produced by the Y lipolytica
strain JM1203 during its growth in a nitrogen-limited medium containing
glucose (at 40 g/l) or glycerol (at 40, 60 or 90 g/l). The culture conditions
are identical to those described for tables 2 and 3.

| | | Fatty acids (%, w/w) | | | | |
|---|---|---|---|---|---|---|
| Substrate | Growth phase | C16:0 | ^{Δ9}C16:1 | C18:0 | ^{Δ9}C18:1 | ^{Δ9,12}C18:2 |
| Glc₀ = 40 g/l | LE or ES (60-90) | 9.7 | 6.2 | 7.0 | 64.8 | 11.8 |
| | S (110-160) | 13.1 | 9.1 | 2.2 | 58.7 | 16.4 |
| Glol₀ = 40 g/l | LE or ES (60-90) | 25.7 | 4.4 | 10.0 | 54.2 | 5.1 |
| | S (110-160) | 21.5 | 8.1 | 13.3 | 47.6 | 9.1 |
| Glol₀ = 60 g/l | LE or ES (60-90) | 24.0 | 3.8 | 15.2 | 52.4 | 4.1 |
| | S (110-160) | 21.2 | 5.8 | 14.7 | 52.0 | 5.8 |
| Glol₀ = 90 g/l | LE or ES (60-90) | 22.7 | 5.0 | 13.4 | 59.7 | 4.0 |
| | S (110-160) | 17.3 | 7.1 | 19.1 | 52.3 | 3.2 |

LE: end of the exponential phase.
ES: start of the stationary phase.
S: stationary phase.

It emerges from these results that the FA composition was modified according to the Glol₀ concentration used and the fermentation time, and that differences were also observed between the growth on glucose and on glycerol. Some differences in the FA profiles were observed between the two strains used, since the culture of the W29 strain resulted in the synthesis of a microbial lipid less rich in oleic acid (^{Δ9}C18:1) and richer in linoleic acid (^{Δ9,12}C18:2) than for the JMY1203 strain. For the equivalent tests on Glol and Glc (initial concentration of 40 g/l) (table 4), the FA composition of the cellular lipids of the W29 strain exhibited a few differences, since the culture on glucose was accompanied by the synthesis of a lipid richer in ^{Δ9,12}C18:2. Differences in the FA composition of the cellular lipids were observed for the JMY1203 strain, for the similar tests on Glol and Glc (table 5); the growth on glucose was accompanied by the synthesis of a lipid richer in ^{Δ9}C18:1 and ^{Δ9,12}C18:2 and less rich in saturated FA. For the W29 strain, the increase in the Glol₀ concentration led to a slight decrease in the concentration of ^{Δ9}C18:1 and a clearer increase in the concentration of ^{Δ9,12}C18:2 (table 4); the reverse tendency was noted for the JMY1203 strain (table 5). The cellular FA composition exhibited an evolution according to the culture time, since, in most of the cases studied, the cellular concentration of ⁴⁹C18:1 had a tendency to decrease with the evolution of the fermentation.

The analysis of the various lipid fractions (N, G+S and P) for Glol₀=90 g/l in the stationary phase showed that, for the JMY1203 strain, the amount of the N fraction is higher than that for the W29 strain (see table 6 hereinafter).

TABLE 6
Distribution of the lipid fractions and fatty acid composition
of the total lipids (T), of the neutral lipids (N), of the glycolipids
plus sphingolipids (G + S) and of phospholipids (P) of the
Y. lipolytica strains W29 and JMY1203 during their growth
in a nitrogen-limited medium containing glycerol (at 90 g/l).
The culture conditions are identical to those described in table
3 above. The sampling point for the lipid analysis is located
in the stationary growth phase (110-160 h).

| | Lipid | %, | | | | | |
|---|---|---|---|---|---|---|---|
| Strain | fraction | w/w | 16:0 | ^{Δ9}16:1 | 18:0 | ^{Δ9}18:1 | ^{Δ9,12}18:2 |
| W29 | T | | 16.8 | 6.8 | 14.5 | 46.7 | 14.9 |
| | N | 73.0 | 15.9 | 7.0 | 12.8 | 48.1 | 13.1 |
| | G + S | 18.9 | 18.1 | 6.1 | 15.1 | 45.1 | 14.1 |
| | P | 8.1 | 15.5 | 6.2 | 13.9 | 45.1 | 17.9 |
| JMY1203 | T | | 17.3 | 7.1 | 19.1 | 52.3 | 3.2 |
| | N | 91.0 | 17.0 | 6.1 | 20.4 | 52.0 | 2.9 |
| | G + S | 2.2 | 17.6 | 7.4 | 18.1 | 50.2 | 3.3 |
| | P | 6.8 | 15.9 | 7.8 | 15.4 | 53.1 | 7.1 |

The analysis of the various lipid fractions (N, G+S and P) showed that the FA composition of these fractions exhibited similarities with the FA composition of the total lipids, whereas the P fraction was slightly richer in polyunsaturated FAs (mainly ^{Δ9,12}18:2) (table 6). The differences in the amounts of N, G+S and P for the W29 and JMY1203 strains can be reflected in the different FA composition of the total lipids of these yeasts (see tables 4 and 5). In the tests with the lowest amounts of total lipids produced (in the case of the W29 strain), greater amounts of the G+S and P fractions (as %, w/w, in the total lipids) (which are more unsaturated) were synthesized.

The yield of citric acid production by the JMY1203 strain was compared with that of several known Y. lipolytica strains cultured under diverse fermentation conditions on supports composed of crude or pure glycerol. The results are given in table 7 hereinafter (legends: n.i.: No information given in the document. *: D=0.009 h⁻¹. $: D=0.021 h⁻¹. §: Total value of the citric acid, the isocitric acid content represents approximately 5%, w/w of the total citric acid).

TABLE 7

| | Citric | Type of | Yield | Type of | |
|---|---|---|---|---|---|
| Strain | acid | glycerol | (g/g) | fermentation | Reference |
| JMY1203 | 57.7^{§} | Crude | 0.92^{§} | Shaken flask | |
| ACA-DC | 33.6 | Crude | 0.44 | Shaken flask | Papanikolaou |
| 50109 | | | | | et al., 2002 |
| Wratislavia | 124.5 | | 0.62 | Batch | Rymowicz et |
| 1.31 | | | | | |
| Wratislavia | 88.1 | | 0.46 | bioreactor | al., 2006 |
| AWG7 | | | | (batch) | |
| Wratislavia K1 | 75.7 | | 0.40 | | |
| NRRL YB-423 | 21.6 | Pure | 0.55 | Shaken flask | Levinson et al., |
| | | | | | 2007 |
| NCIM 3589 | 77.4 | Crude | n.i | | Imandi et al., |
| | | | | | 2007 |
| ACA-DC | 62.5 | | 0.56 | | Papanikolaou |
| 50109 | | | | | et al., 2008 |
| A-101-1.22 | 112.0 | | 0.60 | Batch | Rymowicz et |
| | | | | bioreactor | al., 2008 |
| ACA-YC 5033 | 50.1 | | 0.44 | Shaken flask | André et al., |
| | | | | | 2009 |
| A-101 | 66.5 | Pure | 0.44 | Batch | Rywińska et |
| | 66.8 | Crude | 0.43 | bioreactor | al., 2010a |
| Wratislavia K1 | 53.3 | Pure | 0.34 | | |
| | 36.8 | Crude | 0.25 | | |
| Wratislavia | 126.0 | | 0.63 | Fed-batch | Rywińska et |
| 1.31 | | | | | |
| Wratislavia | 157.5 | | 0.58 | bioreactor | al., 2010b |
| AWG7 | | | | (fed-batch) | |
| Wratislavia | 155.2 | | 0.55 | | |
| 1.31 | | | | | |
| Wratislavia | 154.0 | | 0.78 | Batch | Rywińska and |
| AWG7 | | | | repetition | Rymowicz, |
| | | | | | 2010 |
| N15 | 19.08 | Pure | 0.55 | Shaken flask | Kamzolova et |
| | 98.0 | | 0.70 | Fed-batch | al., 2011 |
| | | | | bioreactor | |
| Wratislavia | 86.5 | | 0.59 | Continuous | Rywińska et |
| AWG7 | | | | bioreactor* | al., 2011 |
| | 63.3 | | 0.67 | Continuous | |
| | | | | bioreactor^{§} | |

The results show that the yield of conversion of glycerol to citric acid (citric acid produced per unit of glycerol consumed) of the JMY1203 strain according to the present invention is higher than that of various Y. lipolytica strains described in the prior art.

iv) Growth of the JMY2900, JMY3776 and JMY4209 Strains in 6% and 9% Glycerol and Glycerol Consumption Over Time

The Y. lipolytica strains JMY2900, JMY3776 and JMY4079 were cultured for 96 h in the Glol 6% medium and the Glol 9% medium. Their growth was determined by measuring optical density at 600 nm (OD₆₀₀). After 96 h of growth, the mass of dry biomass of each strain was measured.

In the Glol 6% medium, the growth of the JMY3776 and JMY4079 strains is approximately 30% to 40% less than the growth of the JMY2900 reference strain, the final biomass after 96 h of culture being 10.56 g/l and 12.36 g/l for JMY3776 and JMY4079 compared with 18.48 g/l for JMY2900. The analysis of the curves of optical density during the growth of the various strains results in the same finding (FIG. 5 and table 8).

In the Glol 9% medium, the growth of the JMY3776 and JMY4079 strains is quite close from the point of view of the optical density; however, the final biomass obtained after 96 h of culture is very different: 18.26 g/l and 10.62 g/l for JMY3776 and JMY4079, compared with 15.24 g/l for JMY2900 (FIG. 5 and table 8). The JMY3776 strain appears to grow much better in the Glol 9% medium than the other strains.

With regard to the glycerol consumption by the various strains in the two media tested, it is possible to note that JMY2900 consumes glycerol more rapidly than JMY3776 and JMY4079 (FIG. 5).

Total exhaustion of the glycerol in the medium occurs at 48 h in the case of the three strains cultured in Glol 6% medium, and at 48 h for JMY2900 and at 72 h for JMY3776 and JMY4079 in Glol 9% medium (FIG. 5). However, the growth continues more or less strongly depending on the strains after exhaustion of the glycerol. It is highly possible that the metabolites secreted by the various strains, as is explained hereinafter, into the culture medium can be used to ensure the growth of said strains after exhaustion of the glycerol (FIG. 5).

TABLE 8
Production of fatty acids, of biomass, of citric acid and of mannitol by the JMY2900,
JMY3776 and JMY4079 strains after 96 h of growth in Glol 6% and 9% medium.

| | | Glol 6% medium (96 h of growth) | | | | | Glol 9% medium (96 h of growth) | | |
|---|---|---|---|---|---|---|---|---|---|
| Parameters | | JMY2900 | JMY3776 | JMY4079 | Parameters | | JMY2900 | JMY3776 | JMY4079 |
| X | g/l | 18.5 | 10.6 | 12.4 | X | g/l | 15.24 | 18.26 | 10.62 |
| Y_{X/S} | g/g | 0.308 | 0.176 | 0.206 | Y_{X/S} | g/g | 0.17 | 0.20 | 0.12 |
| L | g/l | 4.0 | 4.8 | 5.1 | L | g/l | 2.9 | 7.7 | 4.0 |
| Y_{L/X} | g/g | 0.22 | 0.45 | 0.41 | Y_{L/X} | g/g | 0.19 | 0.42 | 0.38 |
| Y_{L/S} | g/g | 0.07 | 0.080 | 0.085 | Y_{L/S} | g/g | 0.03 | 0.086 | 0.044 |
| CA | g/l | 0.26 | 0.06 | 0.05 | CA | g/l | 0.1 | 0.1 | 0.1 |
| Y_{CA/X} | g/g | 0.014 | 0.006 | 0.004 | Y_{CA/X} | g/g | 0.01 | 0.004 | 0.01 |
| Y_{CA/S} | g/g | 0.004 | 0.001 | 0.001 | Y_{CA/S} | g/g | 0.002 | 0.001 | 0.001 |
| Mnt | g/l | 3.2 | 2.7 | 2.9 | Mnt | g/l | 7.5 | 2.0 | 2.8 |
| Y_{Mnt/X} | g/g | 0.17 | 0.26 | 0.23 | Y_{Mnt/X} | g/g | 0.49 | 0.11 | 0.27 |
| Y_{Mnt/S} | g/g | 0.053 | 0.046 | 0.048 | Y_{Mnt/S} | g/g | 0.083 | 0.022 | 0.032 |

Symbols: X, dry biomass; L, lipids; CA, citric acid; Mnt, mannitol; Y_{X/S} or Y_{L/S} or Y_{CA/S} or Y_{Mnt/S}, yield of biomass/lipid/citric acid/mannitol relative to the substrate consumed; Y_{L/X} or Y_{CA/X} or Y_{Mnt/X}, yield of lipid/citric acid/mannitol relative to the biomass produced.

v) Time Course of Citric Acid and Mannitol Production by the JMY2900, JMY3776 and JMY4079 Strains During Growth in Glol 6% or Glol 9% Medium

Whether in the Glol 6% or 9% medium, the citric acid productions of the strains are relatively comparable. Indeed, the JMY3776 and JMY4079 strains produce much more citrate than the JMY2900 strain (FIG. 5). In the two media, the production peak, between 12 and 17 g/l, coincides with the exhaustion of the glycerol. This indicates that these two strains reconsume the citrate once all the glycerol has been consumed. This citrate definitely contributes to the growth of the strains between 48 and 72 h in Glol 6% media and between 72 and 96 h in Glol 9% media. Conversely, the wild-type strain produces only a small amount of citrate, this production being temporary (FIG. 5). On the other hand, JMY2900 appears to be more prompt in producing mannitol, with a production of 11.4 g/l after 48 h of growth in Glol 6% and of 14.8 g/l after 48 h of growth in Glol 9% (FIG. 5). Starting from 48 h, since the medium no longer contains glycerol, the JMY2900 strain reconsumes the mannitol that it has secreted into the culture medium (FIG. 5), thereby no doubt allowing it to be able to continue its growth after 48 h of culture.

After 96 h of growth, the citric acid and mannitol levels are relatively comparable from one strain to the other, except for mannitol which remains 3 times more concentrated (7.5 g/l compared with 2 to 2.8 g/l) in the Glol 9% culture medium of JMY2900 compared with the strains derived from the Δphd1 mutant (table 8 and FIG. 5).

vi) Lipid Production by the JMY2900, JMY3776 and JMY4079 Strains During Growth in Glol 6% or Glol 9% Media

The fatty acid production during the 96 h of culture was analyzed for the three strains in the Glol 6% and 9% media (FIG. 5). It is very clearly apparent that the JMY3776 and JMY4079 strains produce much more fatty acids than the JMY2900 strain. In the Glol 6% medium, the maximum accumulation of fatty acids is reached at 72 h. Said fatty acids then represent 40.5% and 45.5% of the dry weight of the JMY4079 and 3776 strains, respectively, compared with only 20.1% of the dry weight of the JMY2900 strain (FIG. 5). In the Glol 9% medium, the maximum accumulation of fatty acids is also reached after 72 h of culture. However, the fatty acid content is reduced by 10% and 20% in the JMY3776 and JMY4079 strains compared with the Glol 6% medium at the same time (FIG. 5).

It is also interesting to note that the various strains continue to accumulate lipids after exhaustion of the glycerol, this being the case in the two culture media tested (FIG. 5). However, this accumulation is faster in the JMY3776 and JMY4079 strains. Likewise, between 72 and 96 h, in Glol 9% medium, although the major carbon source in the medium is citric acid, the JMY4079 strain continues to strongly accumulate lipids (FIG. 5). This implies that this strain, the growth of which is greatly slowed down, is capable of converting a part of the citric acid into fatty acids. It is highly probable that this is due to the overexpression of the ACL1 and ACL2 genes.

Finally, after 96 h of culture, the lipid production yield reaches 4 and 2.9 g/l for JMY2900, 4.8 and 7.7 g/l for JMY3776 and 5.1 and 4 g/l for JMY4079 in the Glol 6% and Glol 9% media, respectively (table 8). These yields could be greatly improved in the case of fed-batch cultures in a bioreactor. This would make it possible to optimize the growth of the JMY3776 and JMY4079 strains, and also to optimize the conversion of the glycerol into fatty acid while at the same time avoiding the secretion of citric acid or of mannitol into the culture medium.

vii) Fatty Acid Profile of the JMY2900, JMY3776 and JMY4079 Strains During Growth in Glol 6% or Glol 9% Media

The analysis of the profiles of fatty acids synthesized by the JMY2900 strain reveals that the fatty acid composition does not vary (or varies very little) from one medium to the other (cf. table 9 below). The C18:1(n-9) represents close to 55% of the total fatty acids. The C16:0, C16:1(n-7), C18:0 and C18:2(n-6) represent approximately only 11.5%, 5%, 9% and 9% of the total fatty acids. In the case of the JMY3776 and JMY4079 strains, they have a fatty acid content which is quite close, but varies from the JMY2900 strain, and which varies according to the culture medium. Thus, the C18:1(n-9), C16:0, C16:1(n-7), C18:0 and C18:2(n-6) represent approximately 50%, 15% to 20%, 4% to 5%, 7% to 8% and 6.5% of the total fatty acids in Glol 6%. However, in Glol 9%, the proportion of C18:1(n-9) decreases to 45%, to the benefit of C16:0, which represents 25% of the fatty acids.

TABLE 9
Fatty acid profile (as % of total fatty acids) of the JMY2900, JMY3776
and JMY4079 strains after 96 h of growth in Glol 6% and Glol 9% medium.

| | Glol 6% medium (96 h of growth) | | | | Glol 9% medium (96 h of growth) | | |
|---|---|---|---|---|---|---|---|
| | JMY2900 | JMY3776 | JMY4079 | | JMY2900 | JMY3776 | JMY4079 |
| C16:0 | 11.7 | 19.0 | 16.2 | C16:0 | 11.6 | 24.3 | 25.2 |
| C16:1 | 5.2 | 4.7 | 3.9 | C16:1 | 4.9 | 4.8 | 2.9 |
| (n-7) | | | | (n-7) | | | |
| C17:1 | 1.3 | 6.8 | 5.4 | C17:1 | 1.0 | 5.4 | 2.0 |
| C18:0 | 8.8 | 7.3 | 8.1 | C18:0 | 9.4 | 8.6 | 10.4 |
| C18:1 | 54.1 | 49.2 | 51.6 | C18:1 | 58.8 | 45.8 | 44.1 |
| (n-9) | | | | (n-9) | | | |
| C18:2 | 9.0 | 6.0 | 6.6 | C18:2 | 8.9 | 5.7 | 6.2 |
| (n-6) | | | | (n-6) | | | |
| Total | 90.0 | 92.9 | 91.8 | Total | 94.6 | 94.6 | 90.8 |

It is probable that the increased lipid synthesis in the JMY3776 and JMY4079 strains leads to saturation of the elongase, the enzyme responsible for the elongation of fatty acids, including C16:0 to C18:0, but also of the Delta9-desaturase, the enzyme responsible for the desaturation of C18:0 to C18:1(n-9).

### REFERENCES

Anastassiadis S. et al., Appl. Microbiol. Biotechnol., 2002, 60:81-87
André A. et al., Eng. in Life Sci., 2009, 9:468-478
Barth G. and Gaillardin C., 1996, Yarrowia lipolytica, in: Nonconventional yeasts in biotechnology, vol. 1, K. Wolf, K. D. Breunig, and G. Barth (ed.), Springer-Verlag, Berlin, Germany, 313-388
Beopoulos A. et al., Prog. Lipid Res., 2009a, 48:375-387
Beopoulos A. et al. (2009b). Yarrowia Lipolytica as a cell factory for oleochemical biotechnology. Handbook of Hydrocarbon and Lipid Microbiology; Timmis K. N. (ed.); Springer-Verlag: Berlin Heidelberg
Beopoulos A. et al. (2009c). The hydrocarbon-degrading oleaginous yeast Yarrowia lipolytica. Handbook of Hydrocarbon and Lipid Microbiology; Timmis K. N. (ed.); Springer-Verlag: Berlin Heidelberg
Beopoulos A. et al., Appl. Environ. Microbiol., 2008, 74:7779-7789
Beopoulos A. et al., Appl. Microbiol. Biotechnol., 2012, 93:1523-1537
Beopoulos A. et al., Appl. Microbiol. Biotechnol., 2014, 98:251-62
Berninger G. et al., 1993, Eur J Biochem., 216:607-613
Boulton C. A. et al., 1981, J Gen Microbiol., 127:169-176
Browse J. et al., Anal. Biochem., 1986, 152:141-145
Chen F. et al., Acta microbiologica sinica, 2010, 50:1172-1179
Chuang L. T., et al., 2009, Production of functional γ linoleic acid (GLA) by expression of fungal Δ12 and Δ6 desaturases genes in the oleaginous yeast Yarrowia lipolytica. In: Biocatalysis and Agricultural Biotechnology. Ching T Hou, Jei-Fu Shaw eds. PP. 163-180 CRC press, London NY
Dulermo T. and Nicaud J. M., Metab. eng., 2011, 13:482-491
Dulermo T. et al., Biochim Biophys. Acta., 2013, 1831:1486-1495
Einerhand A. W. et al., Mol Cell Biol., 1995, 15:3405-3414
Fakas S. et al., Appl Microbiol Biotechnol., 2006, 73:676-683
Fickers P. et al., Microbiol. Methods, 2003, 55:727-737
Folch J. et al., J. Biol. Chem., 1957, 226:497-509
Fontanille P. et al., Bioresour. Technol., 2012, 114:443-449
Guo X. et al., J. Appl. Microbiol., 2000, 89:107-115
Haddouche R. et al., Appl Microbiol Biotechnol., 2011, 91:1327-1340
Imandi S. B. et al., Bioresour. Technol., 2008, 99:4445-4450
Kamzolova S. V. et al., Food Technol. Biotechnol., 2011, 49:65-74
Lazar Z. et al., J Ind Microbiol Biotechnol., 2013, 40:1273-1283
Levinson W. E. et al., Enz. Microb. Technol., 2007, 41:292-295
Luo Y. S. et al., Arch Biochem Biophys., 2000, 384:1-8
Luo Y. S. et al., Arch Biochem Biophys., 2002, 407:32-38
Madzak C. et al., J Biotechnol., 2004, 109:63-81
Maftahi M. et al., Yeast, 1996, 12:859-868
Makri A. et al., Bioresour. Technol., 2010, 101:2351-2358
Marchler-Bauer A. et al., Nucleic Acids Res., 2011, 39(D):225-229
Mauersberger S. et al. (2003). Tagging genes by insertional mutagenesis in the yeast Yarrowia lipolytica. Laboratory manual on non-conventional yeast in genetics, biochemistry and biotechnology. K. Wolf, K. Breunig and G. Barth. Berlin-Heidelberg-New York, Springer-Verlag: 343-356.
Mauersberger S. et al. (1996). Candida maltosa. Nonconventional Yeasts in Biotechnology. K. Wolf. Berlin, Heidelberg, New York, Springer-Verlag. 1: 411-580
Mlickova K. et al., Appl Environ Microbiol., 2004, 70:3918-3924
Needleman and Wunsch, J. Mol. Biol., 1970, 48:443-453
Nicaud J.-M., Yeast, 2012, 29:409-418
Papanikolaou et al., Antonie van Leeuwenhoek, 2001, 80:215-224
Papanikolaou S. et al., J. Appl. Microbiol., 2002, 92:737-744
Papanikolaou S. et al., Bioresour. Technol., 2008, 99:2419-2428
Papanikolaou S. and Aggelis G., Lipid Technol., 2009, 21:83-87
Papanikolaou S. et al., Eur. J. Lipid Sci. Technol., 2010, 112:639-654
Papanikolaou S. et al., Eur. J. Lipid Sci. Technol., 2011a, 113:1031-1051
Papanikolaou S. et al., Eur. J. Lipid Sci. Technol., 2011b, 113:1052-1073
Ratledge C. (1994). Yeasts, moulds, algae and bacteria as sources of lipids. Technological advances in improved and alternative sources of lipids. B. S. Kamel, Kakuda, Y. London, Blackie academic and professional: 235-291
Rymowicz W. et al., Chem. Pap., 2006, 60:391-394
Rymowicz et al., Appl Microbiol. Biotechnol. 2010, 87:971-979
Rywińska A. et al., Food Technol. Biotechnol., 2009, 47:1-6
Rywińska A. et al., World J. Microbiol. Biotechnol., 2010a, 26:1217-1224
Rywińska A. et al., Electron. J. Biotechnol, 2010b, 13:4
Rywińska A. et al., J. Biotechnol., 2011, 152:54-57
Rywińska A. and Rymowicz V., J. Ind. Microbiol. Biotechnol., 2010, 37:431-435
Sambrook J. et al., Molecular cloning: a laboratory manual, 3^{rd} ed. Cold Spring Harbor Laboratory Press, Cold Spring Harbor, N.Y.
Sherman D. J. et al., Nucleic Acids Res., 2009, 37(Database issue):D550-D554
Tai M. and Stephanopoulos G., Metab. Eng., 2013, 15:1-9
Thevenieau F. et al., Fungal Genet Biol., 2007, 44: 531-42
Thevenieau F. et al. (2009a). Uptake and assimilation of hydrophobic substrates by the oleaginous yeast Yarrowia Lipolytica Handbook of Hydrocarbon and Lipid Microbiology; Timmis K. N. (ed.); Springer-Verlag: Berlin Heidelberg
Thevenieau F. et al. (2009b), Applications of the Non-Conventional Yeast Yarrowia lipolytica, in: Yeast Biotechnology: Diversity and Applications; Satyanarayana T., Kunze G. (eds.); Springer: Netherlands, 589-613
Thevenieau F. & Nicaud J-M., 2013, OCL, 20:D603
Tsigie Y. A. et al., Bioresour. Technol., 2011, 102:9216-9222
Uchiyama H. et al., Eur. J. Biochem., 1982, 125:523-527
Wang H. J. et al., J Bacteriol. 1999a, 181:5140-5148
Wang H. et al., Cell Biochem Biophys., 1999b, 31:165-174
Lazar Z. et al., J Ind Microbiol Biotechnol., 2013 40:1273-1283
Zhang H. et al., Biotechnol Lett., 2013, 35:2091-2098
Zhou J. et al., J Biotechnol., 2012, 161:257-264

1. A mutant yeast strain, wherein:
(a) the expression or the activity of the endogenous 2-methylcitrate dehydratase (EC 4.2.1.79) of said strain is inhibited,
(b) the expression or the activity of at least one of the following proteins of said strain is inhibited:
(i) the endogenous acyl-coenzyme A oxidases (EC 6.2.1.3),
(ii) the endogenous multifunctional beta-oxidation protein (EC 4.2.1.74),
(iii) the endogenous 3-oxoacyl-coenzyme A thiolase (EC 2.3.1.16),
(iv) one or more of the endogenous proteins encoded by a PEX gene involved in yeast peroxisome metabolism,
(v) one or more of the endogenous triacylglycerol lipases (EC 3.1.1.3), and
(vi) the endogenous glycerol-3-phosphate dehydrogenases (EC 1.1.99.5), and
(c) at least one of the following proteins or genes of said strain is overexpressed:
(i) endogenous genes encoding a glycerol-3-phosphate dehydrogenase (NAD(+)) (EC 1.1.1.18),
(ii) an acetyl-CoA carboxylase (EC 6.4.1.2),
(iii) an acyl-CoA:diacylglycerol acyltransferase (EC 2.3.1.20),
(iv) an ATP citrate lyase (EC 2.3.3.8),
(v) a malic enzyme (EC 1.1.1.40),
(vi) an acetyl-CoA synthetase (EC 6.2.1.1),
(vii) a Delta(9)-desaturase (EC 1.14.19.1),
(viii) a Delta(12)-desaturase (EC 1.14.19.6) and
(ix) an invertase (EC 3.2.1.26).

2. The mutant strain as claimed in claim 1, which is an oleaginous mutant yeast strain belonging to the genus selected from the group consisting of Candida, Cryptoccocus, Lipomyces, Rhodosporidium, Rhodotorula, Rhizopus, Trichosporon and Yarrowia.

3. The mutant strain as claimed in claim 2, which belongs to the genus Yarrowia.

4. The mutant strain as claimed in claim 3, which is a Yarrowia lipolytica strain.

5. The mutant strain as claimed in claim 1, selected from the group consisting of:
a strain in which the expression or the activity of the endogenous 2-methylcitrate dehydratase of said strain is inhibited, and the β-oxidation of the fatty acids of said strain is also inhibited,
a strain in which the expression or the activity of the endogenous 2-methylcitrate dehydratase, of one or more endogenous triacylglycerol lipases and of the endogenous multifunctional beta-oxidation protein of said strain is inhibited,
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an endogenous acyl-CoA:diacylglycerol acyltransferase and a glycerol-3-phosphate dehydrogenase (NAD(+)) are overexpressed,
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an acyl-CoA:diacylglycerol acyltransferase, a glycerol-3-phosphate dehydrogenase (NAD(+)) and an ATP citrate lyase are overexpressed,
a strain in which the expression or the activity of the endogenous 2-methylcitrate dehydratase, of one or more endogenous triacylglycerol lipases, of the endogenous multifunctional beta-oxidation protein and of one or more endogenous peroxins of said strain is inhibited, and the endogenous genes encoding an acyl-CoA:diacylglycerol acyltransferase, a glycerol-3-phosphate dehydrogenase (NAD(+)) and an ATP citrate lyase are overexpressed,
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an acyl-CoA:diacylglycerol acyltransferase, a glycerol-3-phosphate dehydrogenase (NAD(+)), an ATP citrate lyase and an acetyl-CoA synthetase are overexpressed,
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an acyl-CoA:diacylglycerol acyltransferase, a glycerol-3-phosphate dehydrogenase (NAD(+)), an ATP citrate lyase, an acetyl-CoA synthetase and a Delta(9)- and/or a Delta(12)-desaturase are overexpressed,
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an acyl-CoA:diacylglycerol acyltransferase, a glycerol-3-phosphate dehydrogenase (NAD(+)), an ATP citrate lyase, an acetyl-CoA synthetase, a Delta(9)- and/or a Delta(12)-desaturase, and an invertase are overexpressed,
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an acyl-CoA:diacylglycerol acyltransferase, a glycerol-3-phosphate dehydrogenase (NAD(+)), an ATP citrate lyase, a malic enzyme and an acetyl-CoA carboxylase are overexpressed, and
a strain in which the expression or the activity of the endogenous 2-methylcitrate dehydratase, of one or more endogenous triacylglycerol lipases and of the endogenous multifunctional beta-oxidation protein of said strain is inhibited, and the endogenous genes encoding an acyl-CoA:diacylglycerol acyltransferase, a glycerol-3-phosphate dehydrogenase (NAD(+)), an ATP citrate lyase, a malic enzyme, an acetyl-CoA synthetase and an acetyl-CoA carboxylase are overexpressed.

6. A process method for obtaining the mutant yeast strain as claimed in claim 1 from a parent yeast strain, the method comprising
a step of mutagenesis of the gene encoding the 2-methylcitrate dehydratase in said parent yeast strain,
one or more steps of mutagenesis in said parent yeast strain resulting in the inhibition of one or more of the endogenous genes encoding the acyl-coenzyme A oxidases (EC 6.2.1.3), the multifunctional beta-oxidation protein (EC 4.2.1.74), the 3-oxoacyl-coenzyme A thiolase (EC 2.3.1.16), at least one of the proteins encoded by the PEX genes involved in yeast peroxisome metabolism, the triacylglycerol lipases (EC 3.1.1.3) and the glycerol-3-phosphate dehydrogenase (EC 1.1.99.5),
and optionally a step of mutagenesis in said parent yeast strain resulting in the overexpression of at least one of the endogenous genes encoding a glycerol-3-phosphate dehydrogenase (NAD(+)) (EC 1.1.1.18), an acetyl-CoA carboxylase (EC 6.4.1.2), an acyl-CoA:diacylglycerol acyltransferase (EC 2.3.1.20), an ATP citrate lyase (EC 2.3.3.8), a malic enzyme (EC 1.1.1.40), an acetyl-CoA synthetase (EC 6.2.1.1), a Delta(9)-desaturase (EC 1.14.19.1), a Delta(12)-desaturase (EC 1.14.19.6) and an invertase (EC 3.2.1.26).

7. A method for increasing the lipid and/or citric acid production of a yeast strain, said method comprising inhibiting the expression or the activity of 2-methylcitrate dehydratase in said yeast strain.

8. The method of claim 7, wherein the expression or of the activity of 2-methylcitrate dehydratase is inhibited by mutagenesis of the gene encoding 2-methylcitrate dehydratase.

9. The method of claim 8, wherein the mutagenesis step results in deletion of the gene encoding 2-methylcitrate dehydratase.

10. The method of claim 7, further comprising:
(a) inhibiting the expression of at least one of:
(i) one or more of the endogenous genes encoding the acyl-coenzyme A oxidases (EC 6.2.1.3),
(ii) the multifunctional beta-oxidation protein (EC 4.2.1.74),
(iii) the 3-oxoacyl-coenzyme A thiolase (EC 2.3.1.16),
(iv) the proteins encoded by the PEX genes involved in yeast peroxisome metabolism,
(v) the triacylglycerol lipases (EC 3.1.1.3) and the glycerol-3-phosphate dehydrogenase (EC 1.1.99.5) and
(b) optionally overexpressing at least one of:
(i) one or more of the endogenous genes encoding a glycerol-3-phosphate dehydrogenase (NAD(+)) (EC 1.1.1.18),
(ii) an acetyl-CoA carboxylase (EC 6.4.1.2),
(iii) an acyl-CoA:diacylglycerol acyltransferase (EC 2.3.1.20),
(iv) an ATP citrate lyase (EC 2.3.3.8),
(v) a malic enzyme (EC 1.1.1.40),
(vi) an acetyl-CoA synthetase (EC 6.2.1.1),
(vii) a Delta(9)-desaturase (EC 1.14.19.1),
(viii) a Delta(12)-desaturase (EC 1.14.19.6) and
(ix) an invertase (EC 3.2.1.26).

11. (canceled)

12. A method for producing lipids and/or citric acid, comprising the step of culturing a mutant yeast strain in which the expression or the activity of the endogenous 2-methylcitrate dehydratase (EC 4.2.1.79) of said strain is inhibited or culturing the mutant yeast strain of claim 1, on an appropriate medium.

13. The method of claim 12, wherein said medium contains at least one of glucose and glycerol as carbon source.

14. A method for producing lipids and/or citric acid, comprising the step of culturing the mutant yeast strain of claim 1 on an appropriate medium.

15. The method of claim 14, wherein said medium contains at least one of glucose and glycerol as carbon source.


SEQUENCE LISTING (26 sequences)

SEQ ID NO: 1 - protein, 520 aa, Yarrowia lipolytica
  DOMAIN (37)..(517): rgion prpD
MRAFRSAANFGAASNIYRKSFTPASIASNRFVSARMSSIMTDNARPNTDKVVQDIADYIHDYKIDSSVAMETARLCFLDTLGCGLEGLKYQQCANIVGPVVPGTIVPNGTKVPGTDYQVDPVRGAFNIGTIIRWLDFNDCWLAAEWGHPSDNLGGILAVADWQTRSAKAGLEGKVFKVKDVLEGMIKAHEIQGGLAIENSFNRVGLDHVVLVKIASTAVVSGMLGLSREQTADAISQAFVDGQSLRTYRHAPNTMSRKSWAAGDATSRAVNLALLVKKGEGGMPSILTAKTWGFYDVLFGGKEFKFQRPYGSYVMENVLFKISFPAEFHAQTACESAMLLHEELKKLGKTSDDIASIKIRTQEAAMRIIDKKGPLHNYADRDHCIQYMVAIPLIHGRLTADDYTDEIASDPRIDALREKMECVEDKRFSEEYHAPDKRYIGNAIEITLKDGTVLDEIEVNYPIGHRQRREEGTPVLLEKFARHLRGRFPEGQVEKILAASNQDIVNMDIDEYVDLYVKKD

SEQ ID NO: 2 - DNA, 18 bp, Artificial Sequence
  Primer
gtcggttggttggatccg

SEQ ID NO: 3 - DNA, 42 bp, Artificial Sequence
  Primer
gcattaccctgttatccctagcgatcctctcaattgggctgg

SEQ ID NO: 4 - DNA, 44 bp, Artificial Sequence
  Primer
gctagggataacagggtaatgccgagatacaggaacgtagtcgg

SEQ ID NO: 5 - DNA, 18 bp, Artificial Sequence
  Primer
cacggggatccacgactc

SEQ ID NO: 6 - DNA, 25 bp, Artificial Sequence
  Primer
gcagtgacacgcgtagtgtagtcgg

SEQ ID NO: 7 - DNA, 22 bp, Artificial Sequence
  Primer
cacgggaatccacgactcctcg

SEQ ID NO: 8 - protein, 491 aa, Artificial Sequence
  Consensus sequence
SSTSIMTDNARPNTDKVVQDIADYIHDYKIDSSVAMETARLCFLDTLGCGLEGLKYQQCANIVGPVVPGTIVPNGTKVPGTDYQVDPVRGAFNIGTIIRWLDFNDCWLAAEWGHPSDNLGGILAVADWQTRSAKTHAGLEGKVFKVKDVLEGMIKAHEIQGGLAIENSFNRVGLDHVVLVKIASTAVVSGMLGLSREQTADAISQAFVDGQSLRTYRHAPNTMSRKSWAAGDATSRAVNLALLVKKGEVGGMPSILTAKTWGFYDVLFGGKEFKFQRRSPYGSYVMENVLFKISFPAEFHAQTACESAMLLHEELKKLGKTSDDIASIKIRTQEAAMRIIDKKGPLHNYADRDHCIQYMVAIPLIHGRLTADDYTDEIASDPRIDALREKMECVEDKRFSEEYHAPDKRYIGNAIEITLKDGTVLDEIEVNYPIGHRQRREEGTPVLLEKFARHLRGRFPEGPDQVEKILAASSNQDIVNMDIDEYVDLYV

SEQ ID NO: 9 - DNA, 46 bp, Artificial Sequence
  Primer
cgcggatcccacaatgtctgccaacgagaacatctcccgattcgac

SEQ ID NO: 10 - DNA, 40 bp, Artificial Sequence
  Primer
caccctaggtctatgatcgagtcttggccttggaaacgtc

SEQ ID NO: 11 - DNA, 43 bp, Artificial Sequence
  Primer
cacggatcccacaatgtcagcgaaatccattcacgaggccgac

SEQ ID NO: 12 - DNA, 44 bp, Artificial Sequence
  Primer
atgcctaggttaaactccgagaggagtggaagcctcagtagaag

SEQ ID NO: 13 - DNA, 29 bp, Artificial Sequence
  Primer
gagagggcgactggattctcttctaccac

SEQ ID NO: 14 - DNA, 29 bp, Artificial Sequence
  Primer
gtggtagaagagaatccagtcgccctctc

SEQ ID NO: 15 - DNA, 31 bp, Artificial Sequence
  Primer
cttcacccaggttggctccaccttcaagggc

SEQ ID NO: 16 - DNA, 31 bp, Artificial Sequence
  Primer
gcccttgaaggtggagccaacctgggtgaag

SEQ ID NO: 17 - DNA, 23 bp, Artificial Sequence
  Primer
cgcggatcccacaatgttacgac

SEQ ID NO: 18 - DNA, 24 bp, Artificial Sequence
  Primer
gcgcctaggctagtcgtaatcccg

SEQ ID NO: 19 - DNA, 36 bp, Artificial Sequence
  Primer
aacgcggatcccacaatggcttcaggatcttcaacg

SEQ ID NO: 20 - DNA, 19 bp, Artificial Sequence
  Primer
gtccaagctcgggaagctg

SEQ ID NO: 21 - DNA, 43 bp, Artificial Sequence
  Primer
ccgttgttagcgatgaggaccttgttgataactgtatgacctc

SEQ ID NO: 22 - DNA, 42 bp, Artificial Sequence
  Primer
gaggtcatacagttatcaacaaggtcctcatcgctaacaacg

SEQ ID NO: 23 - DNA, 21 bp, Artificial Sequence
  Primer
agtatctcatttccgaggctg

SEQ ID NO: 24 - DNA, 42 bp, Artificial Sequence
  Primer
ctggacaccatggctcgtcttgatcccgagtactcctctctc

SEQ ID NO: 25 - DNA, 42 bp, Artificial Sequence
  Primer
gagagaggagtactcgggatcaagacgagccatggtgtccag

SEQ ID NO: 26 - DNA, 40 bp, Artificial Sequence
  Primer
agctatcgataatcctaggtcacaaccccttgagcagctc